(19) United States
(12) Patent Application Publication
(10) Pub. No.: US 2026/0294365 A1
(43) Pub. Date: Oct. 1, 2026

(54) METHOD FOR PROVIDING IMAGE DATA

(71) Applicant: Siemens Healthineers AG, Forchheim (DE)

(72) Inventors: Joerg FREUDENBERGER, Kalchreuth (DE); Martin BATZKE, Nuernberg (DE); Nico JUNG, Bad Blankenburg (DE); Steffen KAPPLER, Hallerndorf-Pautzfeld (DE); Katrin HALL, Eggolsheim (DE); Marcus RADICKE, Veitsbronn (DE); Stefan SPAETH, Bubenreuth (DE); Sebastian GLAWION, Hausen (DE); Ralf NANKE, Neunkirchen am Brand (DE)

(73) Assignee: Siemens Healthineers AG, Forchheim (DE)

(21) Appl. No.: 19/577,730

(22) Filed: Mar. 25, 2026

(30) Foreign Application Priority Data
Mar. 26, 2025 (EP) 25166269.8

(51) Int. Cl. A61B 6/40; A61B 6/00; A61B 6/04; A61B 6/50

(52) U.S. Cl. CPC A61B 6/4021; A61B 6/0414; A61B 6/4452; A61B 6/502

(57) ABSTRACT

A method for providing image data. The method comprises a method step of generating X-ray radiation via an X-ray source. In this case, the X-ray source comprises a cathode for generating an electron beam and an anode. In this case, the electron beam striking the anode forms a focal spot. In this case, the focal spot is moved during an exposure period by deflecting the electron beam on the anode. In this case, the deflection is based on a predetermined control sequence, so that the movement of the focal spot is not continuous in sections. The method comprises a further method step of detecting X-ray radiation via an X-ray detector during the exposure period. The method comprises a further method step of providing the image data based on the detected X-ray radiation.

### CROSS-REFERENCE TO RELATED APPLICATION(S)

The present application claims priority under 35 U.S.C. § 119 to European Patent Application No. 25166269.8, filed Mar. 26, 2025, the entire contents of which is incorporated herein by reference.

### FIELD

The present disclosure relates to a method for providing image data, a method for providing a control sequence, a computer-implemented method for providing a trained function, an optimization system, an X-ray source, an imaging system, a training system, and corresponding computer program products and computer-readable storage media.

One or more example embodiments relates to the field of X-ray imaging, for example, methods for improving image quality in X-ray images with a moving X-ray source.

### RELATED ART

In X-ray imaging, X-ray radiation is typically generated via an X-ray source. This typically penetrates an examination object, which is positioned between the X-ray source and an X-ray detector. The X-ray detector then detects at least part of the X-ray radiation behind the examination object within an exposure period. Image data is generated based on the detected X-ray radiation.

In order to generate the X-ray radiation, the X-ray source typically comprises a cathode and an anode. The cathode typically comprises an emitter. Electrons are emitted from the emitter and accelerated onto the anode. In the following, the terms “the cathode generates electrons” or “the cathode emits electrons” are to be understood as synonymous with the electrons exiting or being emitted from the emitter of the cathode. The acceleration creates an electron beam or an electron cloud. The point at which the electrons strike the anode is called the focal spot. The X-ray radiation is generated, among other things, by the deceleration of the electrons in the focal spot and the excitation of the anode material in the focal spot.

In this case, the dimensions of the focal spot are, among other things, dependent upon its position on the anode. From the perspective of the X-ray detector or the examination object, the dimensions of the focal spot during an exposure period define an effective focus shape for the X-ray radiation. Another factor that plays a role here is whether the X-ray source moves relative to the X-ray detector during the exposure period.

Conventional X-ray systems typically use a fixed focal spot on the anode of the X-ray tube. In imaging techniques such as tomosynthesis, the X-ray source is moved during an exposure period to obtain projection images from different angles. However, this movement of the X-ray source causes blurring or smudging of the focal spot from the perspective of the X-ray detector, resulting in a smudged, enlarged effective focus shape, since the focal spot moves relative to the X-ray detector during the exposure period, which impairs image sharpness.

Previous approaches to reducing this effect include shorter exposure times for the individual projection images or smaller static focal spots. However, these solutions are limited by the required radiation dose and the thermal load capacity of the anode. Furthermore, techniques are known in which the focal spot on the anode is continuously moved synchronously in the opposite direction to the movement of the X-ray source during the exposure time. However, the continuous movement of the focal spot means that it may assume an unfavorable shape and/or size at some positions on the anode. Another approach to reducing smudging of the focal spot from the perspective of the X-ray detector is to keep the position of the X-ray tube stationary during an exposure time. However, this requires accelerating, moving, and decelerating the X-ray tube for the journey from one projection image to the next. This leads to a significantly longer examination time.

### SUMMARY

Known systems have limitations with regard to the achievable image quality, especially with a moving X-ray source. This poses a technical challenge, particularly in applications such as mammography, which require high spatial resolution.

The objective task is to provide a method that ensures optimized image quality.

This objective problem is solved by a method for providing image data, a computer-implemented method for optimizing an effective focus shape, an optimization system, an X-ray source, an imaging system, a training system, and a computer program product according to one or more example embodiments.

### BRIEF DESCRIPTION OF THE DRAWINGS

The properties, features and advantages of this invention described above become clearer and more understandable in context with the figures below and their descriptions. The figures and descriptions are not intended to limit one or more example embodiments and its embodiments in any way.

Identical components in different figures are marked with corresponding reference characters. The figures are generally not true to scale.

In the drawing:

FIG. 1 shows an exemplary embodiment of a method for providing image data,

FIG. 2 shows an exemplary embodiment of a method for providing a control sequence,

FIG. 3 shows an exemplary embodiment of a method for providing a trained function,

FIG. 4a shows an exemplary embodiment of an X-ray source,

FIG. 4b shows a first exemplary embodiment of an X-ray source with a deflection unit,

FIG. 4c shows a second exemplary embodiment of an X-ray source with a deflection unit,

FIG. 5 shows an exemplary embodiment of an imaging system,

FIG. 6 shows an exemplary embodiment of an optimization system,

FIG. 7 shows an exemplary embodiment of a training system,

FIG. 8 shows an exemplary embodiment of a map of dimensions of a focal spot,

FIG. 9a shows a first exemplary embodiment of a movement of a focal spot on an anode,

FIG. 9b shows a resulting movement of the focal spot from the perspective of an X-ray detector according to the first exemplary embodiment,

FIG. 10a shows a second exemplary embodiment of a movement of a focal spot on an anode,

FIG. 10b shows a resulting movement of the focal spot from the perspective of an X-ray detector according to the second exemplary embodiment,

FIG. 11a shows a third exemplary embodiment of a movement of a focal spot on an anode,

FIG. 11b shows a resulting movement of the focal spot from the perspective of an X-ray detector according to the third exemplary embodiment,

FIG. 12a shows a fourth exemplary embodiment of a movement of a focal spot on an anode,

FIG. 12b shows a resulting movement of the focal spot from the perspective of an X-ray detector according to the fourth exemplary embodiment,

FIG. 13a shows a fifth exemplary embodiment of a movement of a focal spot on an anode,

FIG. 13b shows a resulting movement of the focal spot from the perspective of an X-ray detector according to the fifth exemplary embodiment,

FIG. 14a shows a sixth exemplary embodiment of a movement of a focal spot on an anode,

FIG. 14b shows a resulting movement of the focal spot from the perspective of an X-ray detector according to the sixth exemplary embodiment,

FIG. 15a shows a seventh exemplary embodiment of a movement of a focal spot on an anode,

FIG. 15b shows a resulting movement of the focal spot from the perspective of an X-ray detector according to the sixth exemplary embodiment

FIG. 16a shows an eighth exemplary embodiment of a movement of a focal spot on an anode,

FIG. 16b shows a resulting movement of the focal spot from the perspective of an X-ray detector according to the eighth exemplary embodiment,

FIG. 17a shows a first exemplary embodiment of a movement of a focal spot on an anode with a variable focusing, and

FIG. 17b shows a resulting movement of the focal spot from the perspective of an X-ray detector according to the first exemplary embodiment with a variable focusing.

### DETAILED DESCRIPTION

Independent of the grammatical term usage, individuals with male, female or other gender identities are included within the term.

The solution according to one or more example embodiments is described below with reference to both the claimed systems and the claimed methods. Features, advantages, or alternative embodiments may be assigned to the other claimed subjects and vice versa. In other words, claims for the systems may be improved with features that are described or claimed in connection with the methods. In this case, the functional features of the method are embodied by objective units of the system.

One or more example embodiments relates in a first aspect to a method for providing image data. The method comprises, in a first step, generating X-ray radiation via an X-ray source. The X-ray source comprises an anode and a cathode for generating an electron beam. The electron beam striking the anode forms a focal spot. During this process, the focal spot is moved on the anode during an exposure period by deflecting the electron beam. The deflection is based on a predetermined control sequence, so that the movement of the focal spot is not continuous in sections. The method also comprises a step of detecting X-ray radiation via an X-ray detector during the exposure period. The method also comprises a step of providing the image data based on the detected X-ray radiation.

In the method step of generating X-ray radiation, X-ray radiation is generated via the X-radiation source. The X-ray source can be designed as an apparatus which generates X-ray radiation. The X-ray source can be designed so as to generate X-ray radiation with a specific energy or in a specific energy range.

The X-ray source can comprise a cathode and an anode. The cathode can comprise an emitter and a focusing head. The cathode can be designed as an electron source. In other words, electrons can be released or emitted from the cathode. In particular, in this case, the electrons are released or emitted from the emitter. The electron beam is formed by accelerating the electrons released from the cathode onto the anode. The electron beam can also be formed by an individual electron or an electron cloud. In particular, the electron beam can also comprise only some of the electrons released from the cathode. The cathode can be designed, for example, as a hot cathode, dispenser cathode, or field emission cathode. The anode can be designed as a target for the electron beam. The material from which the anode is formed may comprise a material with a high atomic number, such as for example tungsten.

The electron beam can be designed as a directed stream of electrons. The electron beam can be generated by accelerating the electrons emitted from the cathode in an electric field. The electric field can be applied in particular in the form of an accelerating voltage between the anode and the cathode.

The electron beam striking the anode can form a focal spot. The focal spot can be defined as the region on the anode in which the electrons of the electron beam strike the anode and generate X-ray radiation. The size and shape of the focal spot can influence the spatial resolution of the image data generated or of a projection image comprised thereof.

The focal spot can be moved during an exposure period by deflecting the electron beam on the anode. The exposure period can be defined as the time period in which X-ray radiation is detected by the X-ray detector for imaging purposes. The electron beam can be deflected, for example, by electric or magnetic or electro-magnetic fields.

The deflection of the electron beam is based on the determined control sequence. The control sequence can be designed as a sequence of control commands or parameters which specify the movement or the position of the focal spot or the deflection of the electron beam. The control sequence can comprise, for example, deflection values which define the strength and/or direction of the deflection of the electron beam at specific points in time.

The electron beam can be deflected by a deflection unit. This can, for example, generate electric or magnetic or electro-magnetic fields in order to deflect the electron beam.

The movement of the focal spot may not be continuous in sections. This means that the movement of the focal spot may be interrupted or jump, or may move at a varying speed rather than following a smooth, continuous movement. The movement of the focal spot on the anode during the exposure time is referred to as the trajectory.

A further method step involves detecting X-ray radiation via an X-ray detector during the exposure period. An X-ray detector can be designed as an apparatus which converts the X-ray radiation into an electrical signal. The X-ray detector can be designed, for example, as a planar detector, line detector, or single-pixel detector.

When detecting the X-ray radiation, at least some of the X-ray radiation generated is detected via the X-ray detector. Another part of the X-ray radiation generated may not reach the X-ray detector due to scattering, absorption, or the direction of the emitted X-rays, and therefore cannot be detected by the detector.

An examination object can be arranged in particular between the X-ray source and the X-ray detector. In this case, the examination object can be an animal or an object. At least a partial region of the examination object is projected onto the X-ray detector via the X-ray radiation. In this case, the image data comprises at least one projection image of the examination object. When the perspective of the X-ray detector on the focal spot is referred to below, this is to be understood as synonymous with the perspective of the examination object on the focal spot.

The focal spot from the perspective of the X-ray detector is also referred to below as an effective focus and its dimensions as an effective focus shape.

In an optional embodiment, the X-ray radiation can be detected in a pulsed manner. In other words, the X-ray detector can be read multiple times during the exposure time.

A further method step involves providing image data. The image data can be provided by a computing unit. This can process the X-ray radiation detected by the X-ray detector and generate the image data therefrom. The image data can be available, for example, as a two-dimensional projection image or as a re-constructed three-dimensional data record. The image data can be available, for example, as two-dimensional matrices of pixel values or three-dimensional matrices of voxel values.

In the optional case of a pulsed or multiple readout of the X-ray detector, the image data can comprise a combination of the readout data. For this purpose, the readout data can be offset against each other in relation to the image data. Alternatively, the image data can comprise the individual readout data records of the pulsed readout.

In particular, the method can be computer-implemented. In other words, individual or all method steps of the method can be performed via a computer or its components.

The proposed method can offer the advantage that by moving the focal spot in a targeted manner, it is possible to optimize an effective focus shape of X-ray radiation from the perspective of the X-ray detector and thus improve the image quality. In addition, the movement of the focal spot which is not continuous in sections on the anode can render it possible to adapt in a more flexible manner to specific imaging requirements.

In one embodiment of the method, the control sequence can comprise discrete deflection values.

Discrete deflection values can be understood as individual values which are separate from one another and which define the deflection of the electron beam at specific points in time or positions. The discrete deflection values can comprise, for example, deflection voltages, deflection currents or deflection angles for deflecting the electron beam. Alternatively, the discrete deflection values can comprise specific positions of the focal spot on the anode.

In particular, the deflection values can be designed so as to control a deflection unit for deflecting the electron beam.

The specific positions of the focal spot on the anode can be specified as x-y coordinates or polar coordinates. The deflection angle can be defined as an angle relative to a zero position of the electron beam. In the zero position, the electron beam is not deflected.

The control sequence with discrete deflection values can be designed so as to allow the focal spot to jump between defined positions on the anode rather than to specify a continuous movement. Alternatively, the electron beam can be moved continuously at least between individual deflection values of the control sequence.

The discrete deflection values can be stored in a list or table and processed sequentially in order to control the movement of the focal spot.

The control sequence can alternatively specify a functional deflection value-time dependency which is defined by the discrete deflection values.

The use of discrete deflection values in the control sequence can render it possible to control the focal spot movement in a precise manner. As a result, the effective focus shape of the X-ray radiation at the X-ray detector can be optimized over the entire exposure period. The discrete deflection values can be selected, for example, in such a manner that the focal spot assumes preferred positions with favorable focal spot dimensions.

The control sequence with discrete deflection values can advantageously render it possible to implement the focal spot movement in a simple and efficient manner. The discrete values can be used directly as digital control signals for the deflection unit so as to deflect the electron beam and this can simplify the actuation process.

In a further embodiment of the method, the control sequence can specify a type and/or speed of the deflection of the electron beam.

The type of the deflection can relate to the direction and/or shape of the deflection and thus the resulting movement of the focal spot on the anode. For example, the control sequence can specify a deflection which results in the focal spot performing a linear, curved or spiral-shaped movement on the anode. In particular, the type of deflection can specify how the focal spot is to be moved in the movement resulting from the deflection between two discrete deflection values. In other words, the type of the deflection can specify how the focal spot is to be moved in the movement resulting from the deflection between two successive deflection positions, wherein the successive deflection positions are determined by two successive deflection values of the control sequence. In this case, the movement can be a jump, for example. Alternatively, the movement can be interpolated between the two deflection positions. Alternatively, the movement can be determined by a sine/cosine function. Alternatively, the movement can be determined by a periodic function.

The speed of the deflection can relate to the speed at which the deflection of the electron beam is varied and at which the focal spot is moved on the anode as a result. The control sequence can specify, for example, that the deflection of the electron beam is varied continuously or not continuously and that the focal spot moves at a constant speed or at a variable speed as a result.

By specifying the type and/or speed of the deflection via the control sequence, it is possible to control the movement of the focal spot in a precise manner. This renders it possible to improve the image quality.

In a further embodiment of the method, the control sequence can comprise two maximum deflection values.

The two maximum deflection values can each define an opposite maximum deflection of the electron beam.

The maximum deflection values can specify the greatest possible deflection of the electron beam in opposite directions on the anode. The resulting deflection positions of the focal spot are those according to the trajectory of the focal spot at the points furthest away from one another on the anode. “Opposite directions” relates in this case to the deflection angle from the perspective of the zero position.

The maximum deflection values can occur multiple times in the control sequence in order to render it possible to repeat the movement of the focal spot between the maximum positions.

A technical advantage of specifying two maximum deflection values in the control sequence may exist in the fact that it is possible to use the entire possible deflection range of the electron beam or of the focal spot.

In a further embodiment of the method, the control sequence can comprise at least one further deflection value which lies between the maximum deflection values.

The at least one further deflection value can lie spatially and/or temporally precisely between the two maximum deflection values. Alternatively, it can lie spatially and/or temporally closer to one of the two maximum deflection values. Alternatively, it can lie spatially closer to one maximum deflection value and temporally closer to the other maximum deflection value.

When the electron beam is deflected at the point in time 1 according to the one maximum deflection value, at the point in time 2 according to the other deflection value and at the point in time 3 according to the other maximum deflection value, “temporal spacing” means the time between the point in time 1 and the point in time 2 or between the point in time 3 and the point in time 2. In this case, according to the example, the points in time 1, 2 and 3 do not need to be arranged in this sequence temporally one after the other but rather can assume any temporal sequence.

In this case, the spatial spacing is defined by the spatial spacing of the different positions of the focal spot on the anode which the focal spot assumes in dependence upon the different deflection values.

In particular, a multiplicity of further deflection values can lie between the maximum deflection values. These further deflection values can be distributed, for example, equidistant between the maximum deflection values or have a different, non-uniform or not equidistant distribution. The number of further deflection values can be, for example, 1, 2, 3, 4, 5, 10, 20, 50 or more.

The use of further deflection values between the maximum deflection values can render possible a finer control of the focal spot movement. This can lead to an improved optimization of the effective focus shape over the entire exposure period and thus lead to an improved image quality.

In a further embodiment of the method, the X-ray source can be moved during the exposure period according to a planned movement.

The planned movement of the X-ray source can comprise a predetermined path along which the X-ray source moves during the exposure period. This path can be, for example, a linear, curved or circular path. The movement of the X-ray source can be performed continuously or in steps.

The movement of the X-ray source can be used so as to detect different projection images of the examination object and is arranged between the X-ray source and the X-ray detector. For this purpose, multiple image data items can be provided in multiple exposure periods as described above while the X-ray source is being moved. This can be of advantage in particular in tomographic imaging techniques such as computed tomography or tomosynthesis. The movement of the X-ray source renders it possible to record projection images of the examination object from different angles, which renders possible a three-dimensional reconstruction of the examination object.

By taking into account the planned movement of the X-ray source when determining the control sequence, it is possible to maintain that the deflection of the electron beam is adapted to the movement of the X-ray source or coordinated therewith. For example, the deflection can be controlled in such a manner that the focal spot remains at a specific position relative to the X-ray detector for a part of the exposure period, even when the X-ray source moves.

Alternatively, the deflection can be controlled in such a manner that the focal spot describes a specific trajectory which is relative to the X-ray detector and deviates from the movement of the X-ray source.

A technical advantage of this embodiment may exist in the fact that by combining the movement of the X-ray source with the deflection of the electron beam, it is possible to achieve an improved spatial resolution and a reduced radiation exposure for the examination object. The planned movement of the X-ray source in conjunction with the optimized focal spot movement can lead to an improved image quality and an improved diagnostic significance of the image data generated. The control sequence can be adapted in such a manner that distortions or blurring that could be caused by the movement of the X-ray source are minimized. In addition, it is possible to accelerate the imaging during a tomosynthesis, since the X-ray source can be moved quicker during the exposure time possibly due to the movement of the focal spot without significantly impairing image quality.

In a further embodiment of the method, a speed of the movement of the focal spot generated by the deflection of the electron beam can be different at least during a part of the exposure period to a speed of the planned movement of the X-ray source.

A speed of the movement of the X-ray source can be defined as a rate or a tempo at which the position of the X-ray source changes relative to a fixed reference point, for example, to the X-ray detector. The speed of the movement of the X-ray source can be specified in units such as millimeters per second or degrees per second.

The movement or change of the deflection of the electron beam and thus the movement of the focal spot can refer to the movement of the focal spot on the anode of the X-ray source which is generated by the deflection of the electron beam according to the control sequence. This movement can be understood as the trajectory of the focal spot on the anode during the exposure period.

The speed of the movement of the deflection of the electron beam and thus the speed of the movement of the focal spot on the anode can be different at least in sections or at least during a part of the exposure period to the speed of the movement of the X-ray source. This means that the focal spot on the anode can move at least sometimes at a different speed than the X-ray source itself. For example, the speed of the movement of the deflection of the electron beam or the movement of the focal spot on the anode can be quicker, slower or in another direction than the speed of the movement of the X-ray source.

These different speeds render possible a flexible control of the effective focus shape of the X-ray source from the perspective of the X-ray detector. By adapting the speed of the movement of the deflection relative to the speed of the movement of the X-ray source, it is possible to optimize the exposure of the X-ray detector.

In a further embodiment, the movement of the focal spot generated by the deflection of the electron beam can be performed at least partially in the direction of the planned movement of the X-ray source.

In other words, the movement of the deflection of the electron beam or of the focal spot on the anode can have in this case a component which runs parallel to the movement direction of the X-ray source.

The movement of the deflection of the electron beam or the movement of the focal spot on the anode in the direction of the planned movement of the X-ray source can be performed continuously or discontinuously. In the case of a continuous movement, the focal spot can continuously follow the planned movement of the X-ray source. In the case of a discontinuous movement, the focal spot can be displaced in discrete steps in the direction of the planned movement of the X-ray source.

The speed of the movement of the deflection of the electron beam or of the focal spot on the anode in the direction of the planned movement of the X-ray source can be constant or variable.

In addition to the movement in the direction of the X-ray source, the deflection of the electron beam or of the movement of the focal spot on the anode can also optionally comprise movement components in other directions, in order, for example, to ensure an optimum coverage of the region to be examined or in order to distribute thermal loads on the anode. Optionally, the movement of the focal spot in the direction of the X-ray source movement can be combined with other movement components of the focal spot. It is thus possible to superimpose an oscillating movement perpendicular to the movement direction of the X-ray source. This can lead in particular to a higher load capacity and thus under certain conditions to a longer serviceable like of the anode.

A technical advantage of this embodiment may exist in the fact that the coordinated movement of the focal spot and the X-ray source can render it possible to improve image quality while simultaneously optimizing the effective focus shape. The adaptation of the focal spot movement to the X-ray source movement can lead to a more uniform exposure of the examination object and thus reduce artifacts in the resulting image data.

In a further embodiment of the method, the control sequence can comprise at least four different deflection values. The control sequence can comprise each of the deflection values twice. The electron beam can be deflected in such a manner that it is moved continuously between at least two deflection values following one another according to the control sequence opposite the direction of the movement of the X-ray source. The electron beam can be deflected in such a manner that it jumps between at least two further deflection values following one another according to the control sequence in the direction of the movement of the X-ray source.

The control sequence can comprise each of the deflection values twice. This can render it possible for the focal spot to assume a specific position on the anode multiple times during the exposure period.

The electron beam can be deflected in such a manner that it is moved continuously between at least two deflection values following one another according to the control sequence opposite the direction of the movement of the X-ray source. A continuous movement can be designed as a continuous, uninterrupted movement without abrupt jumps.

The electron beam can be deflected in such a manner that it jumps between at least two further deflection values following one another according to the control sequence in the direction of the movement of the X-ray source. A jump can be designed as an abrupt, discontinuous change in position of the electron beam.

The continuous movement of the electron beam in the opposite direction of the movement of the X-ray source can be used to reduce the effective speed of the focal spot relative to the examination object. This can contribute to improving the image quality, since the movement blur can be reduced.

The jump of the electron beam in the direction of the movement of the X-ray source between successive deflection values can render it possible to move the focal spot rapidly into a new position in order to use a larger region of the anode for generating the X-ray radiation. This can contribute to reducing the thermal load on the anode. In addition, it is possible to jump into regions in which the shape of the focal spot is advantageous, in particular narrow, for example.

The combination of a continuous movement and abrupt movement of the electron beam can render it possible to use the advantages of both types of movement. The continuous movement can ensure an improved image quality, whereas the abrupt movement can render it possible to optimize the dimension of focal spot.

In a further embodiment of the method, the method can be repeated for at least two exposure periods during the movement of the X-ray source.

By repeatedly performing the method for multiple exposure periods, it is possible to generate multiple projection images of the examination object in particular from different angles if the X-ray source is moved during this process. This can be advantageous especially for tomographic imaging techniques, such as tomosynthesis. The image data thus comprises at least two projection images of the examination object. Alternatively, image data is provided for each exposure period and comprises in each case a projection image.

It is possible to provide between the individual exposure periods a pause in which X-ray radiation is not generated. This can be used to reduce the thermal load on the anode. In addition, it is possible in this manner to reduce the applied dose. Alternatively, the exposure periods can also directly follow one another in order to render it possible to perform the imaging as continuously as possible.

By repeatedly performing the method for multiple exposure periods, it is possible to reconstruct or determine a three-dimensional representation of the examination object from the multiple projection images.

In a further embodiment of the method, the planned movement of the X-ray source can be continuous.

A continuous movement of the X-ray source can be designed as a uniform, uninterrupted movement of the X-ray source during the exposure period. The continuous movement can comprise, for example, a uniform rotation or translation of the X-ray source.

The continuous movement of the X-ray source can be designed as a circular movement, a linear movement, a curved movement or any combination of the previously mentioned movements. In the case of a circular movement, the X-ray source can rotate, for example, around a fixed middle point. The linear movement can comprise a displacement of the X-ray source in a straight line along a predefined axis. In the case of a curved movement, the X-ray source moves in a curved path which does not have a fixed middle point. This path may be elliptical, for example.

The continuous movement of the X-ray source can be performed at a constant or variable speed.

During the continuous movement of the X-ray source, the method for providing image data can be performed for multiple successive exposure periods. This renders it possible to record a series of projection images of the examination object from different angles.

By continuously moving the X-ray source during the exposure period, it is possible to achieve an improved spatial coverage of the examination object. This can lead to an improved image quality and a reduced radiation exposure for the examination object. In addition, continuous movement is more predictable and can thus be taken into account more easily when generating the image data or when reconstructing three-dimensional images of the examination object.

In a further embodiment of the method, the control sequence can comprise at least two discrete deflection values following one another. The electron beam can be deflected in such a manner that it jumps between at least two discrete deflection values following one another according to the control sequence.

“Discrete deflection values following one another” means that the deflection values are arranged in a specific order one after the other in the control sequence. A defined temporal or spatial spacing can be between two discrete deflection values following one another.

The electron beam can be deflected in such a manner that it jumps between at least two discrete deflection values following one another according to the control sequence. “The electron beam jumps” means that the electron beam is moved rapidly and directly from a deflection defined by a first discrete deflection value to a deflection defined by a second discrete deflection value without performing any temporally relevant intermediate deflections. The focal spot on the anode thus likewise jumps from one position to the other position without assuming temporally relevant intermediate positions, wherein the positions depend directly on the deflection values.

An abrupt change in the deflection voltage or the deflection current in the deflection unit causes the electron beam to jump.

The number of discrete deflection values in the control sequence can vary and be adapted to the specific requirements of the application. It is possible to use, for example two, three, four or more discrete deflection values.

The temporal spacings between the jumps can be constant or variable. Variable temporal spacings can be used in order to optimize the dwell time of the focal spot at specific positions.

A discontinuous movement of the focal spot on the anode can be generated by jumping between discrete deflection values. This can be advantageous in order to actuate specific regions of the anode in a targeted manner or in order to achieve a desired effective focus shape of the X-ray radiation from the perspective of the X-ray detector.

In a further embodiment of the method, the electron beam can be deflected in such a manner that the movement of the focal spot is at least partially continuous.

The movement of the focal spot is generated by the deflection of the electron beam. The deflection of the electron beam can be controlled in this case in such a manner that the focal spot moves at least in sections continuously on the anode.

A continuous movement can be designed as a movement in which the focal spot moves without interruption or abrupt changes from one position on the anode to another position on the anode. In the case of a continuous movement, the position of the focal spot can be described at any point in time by a continuous function. In contrast thereto, a discontinuous movement can have jumps or interruptions.

The continuous movement of the focal spot can be achieved, for example, by a continuous change of the deflection voltage or the deflection current. The control sequence can comprise corresponding continuous deflection values in order to generate a continuous movement of the focal spot. Alternatively, the control sequence can comprise a functional connection between time and deflection value.

The continuous movement of the focal spot can be combined with discrete jumps of the focal spot. For example, the focal spot can move continuously between two discrete positions on the anode and then to a third discrete position on the anode.

The continuous movement of the focal spot can take place in different directions. The focal spot can move, for example, in a linear manner, in a circular manner or along a more complex trajectory.

The speed of the continuous movement of the focal spot can be varied.

An at least partially continuous movement of the focal spot can offer multiple technical advantages. It can lead to a more uniform exposure of the X-ray detector, which can improve the image quality. Furthermore, a continuous movement of the focal spot can render possible a finer control of the effective focus shape of the X-ray radiation from the perspective of the X-ray detector, which can lead to an improved resolution of the generated image data.

In a further embodiment of the method, the method can comprise in addition the following method step: focusing the electron beam around a zero position of the focal spot via a focusing unit.

A focusing unit can be designed as a component which is configured so as to focus an electron beam. The focusing unit can be designed, for example, as an electro-static or magnetic focusing unit. An electro-static focusing unit can comprise, for example, one or more electrodes, to which it is possible to apply a voltage in order to focus the electron beam. The electro-static focusing unit can be designed as part of a focusing head of the X-ray source. A magnetic focusing unit can comprise, for example, one or more coils through which a current can flow in order to generate a magnetic field which focuses the electron beam. The magnetic focusing unit can be part of the X-ray source.

The zero position of the focal spot can be designed as the particular position on the anode at which the electron beam strikes if deflection does not take place.

The focusing of the electron beam around the zero position of the focal spot can be designed as a process in which the focusing unit concentrates the electron beam in such a manner that, in the zero position and optionally also in regions around the zero position, it generates a smallest possible focal spot on the anode. In this case, the focusing of the electron beam can vary in dependence upon the position of the focal spot on the anode.

The focusing of the electron beam can be combined with the deflection of the electron beam. For example, the focusing of the electron beam can be adapted during the deflection of the electron beam in order to optimize the size of the focal spot for different deflection positions.

A technical advantage of focusing the electron beam around the zero position via a focusing unit may exist in the fact that it is possible to optimize the size of the focal spot in the zero position, which can lead to an improved resolution of the generated X-ray radiation. In addition, by combining focusing and deflecting, it is possible to achieve an optimized size of the focal spot over the entire deflection region.

In a further embodiment of the method, the focusing with the focusing unit can be less, the further away the focal spot is moved from the zero position by deflecting the electron beam.

The further away the focal spot from the zero position, the less intense the focusing by the focusing unit can be. This may be due to the fact that the deflection of the electron beam itself already leads to a focusing. The strength of the focusing by the focusing unit can therefore be adapted in dependence upon the deflection of the electron beam.

If the focal spot is moved away from the zero position, it is possible to reduce the focusing by the focusing unit in steps. This can take place, for example, by reducing the voltage applied to the focusing unit or the current flowing through the focusing unit. It is possible to reduce the focusing proportionally to the increasing deflection of the electron beam and thus proportionally to the spacing of the focal spot from the zero position.

By adapting the focusing in dependence upon the deflection, it is possible to achieve a uniform size of the focal spot over the entire deflection range. This can contribute to improving the image quality, since variations of the size of the focal spot can lead to blurring and/or distortion in the image data.

According to an optional embodiment, it is possible to vary a cathode current and/or the acceleration voltage around the zero position of the focal spot.

In particular, when the focal spot is at the zero position, it is possible to increase the acceleration voltage, since in this manner the dimensions of the focal spot can be reduced. In particular, the acceleration voltage can be reduced continuously with increasing deflection of the electron beam.

Alternatively, when the focal spot is at the zero position, it is possible to reduce the acceleration voltage. It is possible in this manner to reduce the influence of the focal spot in this region on the effective focus shape. In this manner, the influence of the focal spot on the effective focus shape from the perspective of the X-ray detector, when it is particularly large, is as small as possible. The narrower the focal spot, in other words, the further the focal spot is deflected from the zero position, the greater the acceleration voltage can be, the greater also is the influence on the effective focus shape.

Alternatively or additionally, when the focal spot is at the zero position, it is possible to reduce the cathode current. The number of electrons released from the cathode or the emitter in the cathode is dependent upon the cathode current. By reducing the cathode current, fewer electrons are released from the cathode. Consequently, the focal spot at the zero position has less influence on the effective focus shape from the perspective of the X-ray detector over the entire exposure time. In particular, the cathode current can be continuously increased with increasing deflection of the electron beam.

In this manner, the effective focus shape can be further optimized. In particular, it is possible to reduce disturbing influences on the effective focus shape.

In a further optional embodiment, the cathode current and/or the acceleration voltage can be varied, while the focal spot assumes a discrete position on the anode.

In particular, the acceleration voltage can be varied in such a manner that it is increased when the focal spot assumes an optimum position from the perspective of the X-ray detector. The optimum position can be, for example, when the focal spot is positioned in the middle in the effective focus. When the focal spot is moved away from this optimum position relative to the X-ray detector, for example by moving the X-ray tube and/or by deflecting the electron beam, it is possible to reduce the acceleration voltage.

In particular, the cathode current can be varied in such a manner that it is increased when the focal spot assumes an optimum position from the perspective of the X-ray detector. The optimum position can be, for example, when the focal spot is positioned in the middle in the effective focus. When the focal spot is moved away from this optimum position relative to the X-ray detector, for example by moving the X-ray tube and/or by deflecting the electron beam, it is possible to reduce the cathode current.

By varying the acceleration voltage and/or the cathode current, the influence of the focal spot on the effective focus can be increased at specific times or at specific positions. In this manner, the effective focus shape can be further optimized.

One or more example embodiments relates in a second aspect to a computer-implemented method for providing a control sequence. The method comprises a step of receiving a map of dimensions of a focal spot. In this case, the focal spot is formed by an electron beam striking an anode of an X-ray source. In this case, the dimensions depend upon a deflection of the electron beam. The method also comprises a method step of determining a control sequence for deflecting the electron beam in dependence upon the map. In this case, the control sequence is determined in such a manner that an effective focus shape of X-ray radiation emitted by the focal spot is optimized over an exposure period with regard to the dimensions of the focal spot. The method also comprises a method step of providing the control sequence.

With regard to the definitions of features which are already comprised in the method described above, reference is made to the description above.

Receiving a map of focal spot dimensions can be designed as receiving or importing information regarding the size and/or shape of the focal spot at different positions on the anode. The map can be in the form of a data structure, a table or any other suitable representation. The positions of the focal spot on the anode depend directly on a deflection of the electron beam.

When determining the control sequence, the control sequence is determined or calculated in dependence upon the map.

The dependency upon the map can be designed as a relationship or connection between the information contained in the map and the control sequence to be determined. The dependency may exist, for example, in the fact that areas with smaller focal spot dimensions are preferentially targeted.

The control sequence can be determined in such a manner that the effective focus shape is optimized with regard to focal spot dimensions over the entire exposure period. In this case, it is possible, for example, to aim for a smallest possible effective focus shape of the X-ray radiation from the perspective of the X-ray detector in order to achieve a high resolution. At the same time, a uniform load on the anode can be taken into account. Alternatively or additionally, it is possible to aim for a focus shape that is as uniform as possible. A uniform focus shape can refer in particular to the most uniform intensity distribution possible, for example a Gaussian intensity distribution.

Providing the control sequence can be designed as outputting, transmitting or making available the specific control sequence for a further use. The control sequence can be transmitted, for example, to a deflection unit of the X-ray source.

The described method renders possible an optimized control of the movement of the focal spot on the anode or the deflection of the electron beam in order to improve the effective focus shape of the X-ray beam from the perspective of the X-ray detector. This can improve the image quality of X-rays without increasing the radiation exposure for the patient.

In one embodiment of the method, the control sequence provided is designed for use in a method described above for providing image data.

The use of the control sequence in the above described method for providing image data can mean that the optimized control sequence which has been determined by the method for providing the control sequence is used as an input for the deflection unit of the X-ray source in a method described above for providing image data.

Forming the control sequence for use in the method for providing image data can include that the format and the structure of the control sequence is selected such that it is designed for deflecting the electron beam. In particular, the format and the structure of the control sequence can be designed in such a manner that they are compatible with the deflection unit in the X-ray source. This can comprise, for example, the adaptation of the temporal resolution, the value range or the data format of the control sequence.

By using the optimized control sequence in the method for providing image data, it is possible to achieve an improved image quality. The optimized control sequence renders possible targeted control of the focal spot movement, as a result of which the effective focus shape can be optimized over the entire exposure period. This can lead to a reduction in movement blurs and an improvement in the spatial resolution of the generated image data.

In addition, by integrating the optimized control sequence into the method for providing image data, it is possible to use the X-ray source efficiently. The targeted control of the focal spot movement can lead to a more uniform load on the anode which can increase the serviceable life of the X-ray source.

A further advantage may exist in the fact that by using the optimized control sequence, it is possible to adapt to difference imaging situations and examination objects. The control sequence can, for example, be adapted to different movements of the X-ray source or different examination objects in order to achieve in each case optimum imaging results.

In a further embodiment, the method comprises a method step of receiving a planned movement of the X-ray source during the exposure period. In this case, the planned movement of the X-ray source is taken into account when determining the control sequence.

Receiving the planned movement of the X-ray source can comprise recording and/or receiving the planned movement. The planned movement can be received, for example, via an interface. The planned movement can be in the form of a position-time dependency of the position of the X-ray source, for example, with regard to the X-ray detector.

Taking into account the planned movement of the X-ray source when determining the control sequence can mean that the deflection of the electron beam is adapted to the movement of the X-ray source. For example, the deflection can be controlled in such a manner that it counteracts or compensates for the movement of the X-ray source in order to achieve a specific effective focus shape. Alternatively, the deflection of the electron beam can be determined in such a manner that the focal spot moves relative to the examination object or to the X-ray detector in a predetermined manner while the X-ray source moves.

It is possible to improve the image quality by taking into account the planned movement of the X-ray source when determining the control sequence. The deflection of the electron beam can be adapted optimally to the movement of the X-ray source, as a result of which movement artifacts can be reduced and the effective focus shape can be optimized from the perspective of the X-ray detector over the entire exposure period.

In a further embodiment, the method can also comprise a method step of applying a trained function to the map and optionally the planned movement of the X-ray source. The control sequence is determined in the process.

A trained function can be designed as a mathematical model or an algorithm which has learned, on the basis of training data, to convert specific inputs into desired outputs. The trained function may comprise, for example, an artificial neural network, a decision tree, a random forest, or a support vector machine.

Applying a trained function can be designed as the process in which the trained function processes new, unseen input data in order to generate an output. During applying, the parameters of the function learned during training are used to transform the input data and generate a prediction or classification.

The map and optionally the planned movement of the X-ray source can be designed as input data for the trained function. The trained function can be designed as a machine learning model that has been trained on training data. The machine learning model may comprise, for example, a neural network, a decision tree, a random forest, or a support vector machine.

Determining the control sequence based on the output of the trained function can be designed as a process in which the output generated from the trained function is interpreted and converted into specific control instructions for deflecting the electron beam. This can comprise converting abstract predictions or classifications into temporally resolved deflection values.

It is possible by applying a trained function to determine an optimized control sequence in an efficient and automated manner without having to perform complex manual calculations or optimizations. In this case, the trained function can have implicitly learned to take into account different factors such as the focal spot dimensions and the X-ray source movement in order to achieve an optimum as possible effective focus shape of the X-ray radiation from the perspective of the X-ray detector.

In a further alternative embodiment, determining the control sequence can comprise the following method steps:

In a first step, an optimization function can be created in dependence upon the map and optionally the planned movement of the X-ray source.

In a further step, the control sequence can be determined based on the optimization function.

An optimization function can be designed as a mathematical function which optimizes one or more parameters. The optimization function can be designed to minimize or maximize an objective function. In this context, the optimization function can be designed as so as to optimize the effective focus shape of the X-ray radiation from the perspective of the X-ray detector over the exposure period with regard to the focal spot dimensions.

Creating an optimization function can comprise defining a mathematical function which takes into account parameters and boundary conditions which are to be optimized. The optimization function can be designed, for example, as a cost function or a utility function.

“In dependence upon the map” can mean that the optimization function takes into account the information contained in the map regarding focal spot dimensions for different positions of the focal spot on the anode. The optimization function can be created in such a manner that the focal spot dimensions for different deflections of the electron beam are used as input parameters. In this case, different criteria can be taken into account, such as for example the minimization of the average effective focus size or the maximization of the resolution in specific regions of the image field.

Optionally, the planned movement of the X-ray source can be taken into account when creating the optimization function. This can mean that the optimization function also includes parameters which describe the movement of the X-ray source during the exposure period.

Determining the control sequence based on the optimization function can comprise applying optimization algorithms to the created optimization function. In this case, different optimization methods, such as for example gradient methods, evolutionary algorithms, numerical optimization methods or simulated annealing, can be used. The result of this optimization process can be the control sequence which specifies the optimum deflection of the electron beam over the exposure period.

One advantage of the method may exist in the fact that the control sequence can be adapted optimally to the specific characteristics of the X-ray source used or optionally the planned movement. This renders it possible to achieve an improved image quality.

One or more example embodiments relates in a third aspect to a computer-implemented method for providing a trained function for determining a control sequence for deflecting an electron beam. The method comprises a step of receiving training input data. The training input data comprises a map of dimensions of a focal spot. In this case, the focal spot is formed by an electron beam striking an anode of an X-ray source. In this case, the dimensions depend upon a deflection of the electron beam. The training input data also optionally comprises a planned movement of the X-ray source. The method comprises a further step of receiving training output data. The training output data comprises in this case control sequences with which optimized effective focus shapes of X-ray radiation emitted by the focal spot can be achieved. The method comprises a further step of training the trained function based on the training input data and the training output data. The method comprises a further step of providing the trained function.

With regard to the definitions of features which are already comprised in the methods described above, reference is made to the description above.

Training input data is received in a method step. The training input data comprises a map of dimensions of a focal spot generated by an electron beam striking an anode of an X-ray source for different positions of the focal spot on the anode and optionally a planned movement of the X-ray source.

Receiving can comprise receiving and/or acquiring the training input data. The training input data can be received, for example, via an interface. The interface can be designed as a hardware or software interface, for example as a PCI bus, CAN bus, USB or Firewire.

The map of dimensions of the focal spot can be understood as assigning focal spot dimensions to different positions of the focal spot on the anode. The focal spot dimensions can comprise, for example, the width, length or area of the focal spot. The different positions of the focal spot can be described by coordinates on the anode.

Alternatively, the different positions can be described by deflection values based on which the electron beam can be deflected in such a manner that it strikes the anode at the corresponding position.

Training output data is received in a further method step. The training output data comprises control sequences with which optimized effective focus shapes of X-ray radiation emitted by the focal spot can be achieved. The effective focus shapes are evaluated in this case from the perspective of a fixed reference point, for example an X-ray detector or an examination object.

The training output data can be understood as meaning reference data which represents the desired results for the corresponding training input data. The control sequences for achieving optimized effective focus shapes can be understood as control sequences with which ideal or desired shapes of the focal spot can be achieved from the perspective of the X-ray detector, which are considered optimum for certain applications or imaging conditions.

In a further step, the trained function is trained based on the training input data and the training output data. The trained function may already be pre-trained.

Alternatively, the trained function at the beginning of the training can be a basic function without pre-training.

Training the trained function can be understood as meaning a process in which the parameters of the trained function are adapted in such a manner that they produce the best possible desired training output data for the given training input data. The training can take place, for example via machine learning, such as through the use of neural networks, support vector machines or other machine learning algorithms.

The trained function is provided in a last step.

Providing the trained function can be understood as meaning providing-creating the function for further application. This can take place, for example, by storing the function in a storage device, transferring the function to another system or integrating the function into an existing system.

The trained function can be designed, based on a map of dimensions of a focal spot on an anode and optionally on a planned movement of the X-ray source, to determine a control sequence for deflecting the electron beam. The control sequence can be determined in such a manner that the effective focus shape is optimized with regard to focal spot dimensions over an entire exposure period.

The computer-implemented method for providing a trained function can offer the advantage that a trained function can be provided which is able to determine for different input conditions optimum control sequences for deflecting the electron beam. This can lead to an improved image quality in the X-ray imaging, since the focal spot dimensions and consequently the quality of the X-ray radiation can be optimized.

The trained function which is provided by this method can be used in a method described above for providing a control sequence or in a method described above for providing image data.

In general, a trained function replicates cognitive functions that humans associate with other human minds. In particular, through training based on training data, the trained function may be able to adapt to new circumstances and recognize and extrapolate patterns. Another term for ‘trained function’ is ‘trained model’. In general, parameters of a trained function can be adapted via training. In particular, supervised training, semi-supervised training, unsupervised training, reinforcement learning and/or active learning may be used. Furthermore, representation learning (an alternative term is ‘feature learning’) can be used. In particular, the parameters of the trained function can be adapted iteratively through several training steps. In particular, a specific cost function can be minimized within the training. In particular, the backpropagation algorithm can be used within the training of a neural network. In particular, a trained function may comprise a neural network, a support vector machine, a decision tree and/or a Bayesian network, and/or the trained function may be based on k-means clustering, Q-learning, genetic algorithms and/or association rules. In particular, a neural network may be a deep neural network, a convolutional neural network or a deep convolutional neural network. Furthermore, a neural network may be an adversarial network, a deep adversarial network and/or a generative adversarial network.

By using convolutional neural networks, input images can be processed very efficiently, since a convolution operation based on different kernels can extract different image features, so that by adapting the weights of the convolution kernel, the relevant image features can be found during training. Furthermore, due to the weight distribution in the convolutional kernels, fewer parameters need to be trained, which prevents over-adapting in the training phase and enables faster training or more layers in the network, which improves the performance of the network.

According to one aspect, the trained function can comprise one or more residual networks (ResNet). In particular, a ResNet is an artificial neural network that comprises at least one skip or jump connection which is used to skip at least one layer of the artificial neural network. In particular, a ResNet can be a convolutional neural network that comprises one or more skip connections which in each case skip one or more convolution layers. According to some examples, the ResNets can be represented as m-layer-ResNets, wherein m is the number of layers in the corresponding architecture and according to some examples can assume values of 34, 50, 101 or 152. According to some examples, such a m-layer-ResNet can comprise in each case (m−2)/2 skip connections.

A skip connection can be regarded as a bypass which directs the output of a preceding layer directly via one or more skipped layers to a layer following the one or more skipped layers. Instead of having to adapt a desired image directly, the skipped layers would then have to adapt a residual image that ‘compensates’ for the directly forwarded output.

Adapting the residual image is easier to optimize mathematically than the direct imaging. Furthermore, this mitigates the problem of vanishing/exploding gradients during optimization when training the trained functions: If a skipped layer encounters such problems, its contribution can be skipped by regularizing the directly forwarded output. The use of ResNets therefore has the advantage that much deeper networks can be trained. In the context of the planned provision of a trained function for determining a control sequence for deflecting an electron beam, this has the advantage of more accurate prediction of the control sequence—and thus contributes to improved optimization of the effective focus shape.

A generative adversarial model (abbreviated as GA model) comprises a generative function and a discriminative function, wherein the generative function generates synthetic data and the discriminative function distinguishes between synthetic and real data. On the one, by training the generative function and/or the discriminative function, the generative function is configured to generate synthetic data that is incorrectly classified as real by the discriminative function, while the discriminative function is configured to distinguish between real data and synthetic data generated by the generative function. In terms of game theory, a generative adversarial model can be interpreted as a zero-sum game. The training of the generative function and/or the discriminative function is based in particular on the minimization of a cost function.

By using a GA model, it is possible based on a set of training data to generate synthetic data which has the same characteristics as the training data record. Training the GA model can be based on annotated data (unsupervised learning), so that the effort required to train a GA model is low.

In particular, a recurrent trained function is a trained function whose output depends not only on the input value and the parameters of the trained function adapted by the training process, but also on a hidden state vector, wherein the hidden state vector is based on previous inputs used for the recurrent trained function. In particular, the recurrent trained function can comprise additional storage states or additional structures that incorporate time delays or include feedback loops.

In particular, the structure forming the basis of a recurrent trained function can be a neural network which can be referred to as a recurrent neural network. Such a recurrent neural network can be described as an artificial neural network in which connections between nodes form a directed graph along a temporal sequence. In particular, a recurrent neural network can be interpreted as a directed acyclic graph. In particular, the recurrent neural network may be a recurrent neural network with finite pulse response or a recurrent neural network with infinite pulse response (wherein a network with finite pulse response can be unfolded and replaced by a strictly forward-looking neural network, and an infinite impulse response network cannot be unfolded and replaced by a strictly forward-looking neural network).

In particular, the training of a recurrent neural network can be based on the BPTT algorithm (abbreviation for ‘backpropagation through time’), the RTRL algorithm (abbreviation for ‘real-time recurrent learning’) and/or genetic algorithms.

By applying a recurrent trained function, input data can be used which comprises sequences of variable length. In particular, this means that the method can not only be used for a fixed number of input data records (and must be trained differently for any other number of input data records used as an input), but rather can be used for any number of input data records. This means that the entire set of training data can be used within the training irrespective of the number of input data records contained in different sequences and that training data is not reduced to training data which corresponds to a specific number of successive input data records.

A transformer network is a neural network architecture that generally comprises an encoder, a decoder, or both an encoder and a decoder. In some cases, the encoders and/or decoders consist of multiple corresponding encoding layers and decoding layers. Within each encoding and decoding layer, there is preferably an attention mechanism. The attention mechanism, sometimes referred to as self-attention, relates data elements (such as words or pixels) within a series of data elements to other data elements within this series. The self-attention mechanism enables the model, for example, to examine a group of voxels within a medical image and determine the relative importance of other groups of voxels within this medical image to the examined group of voxels.

In particular, the encoder may be configured so as to transform the input into a numerical representation. The numerical representation may comprise one vector per input token. The encoder may be configured so as to implement an attention mechanism such that each vector of a token is influenced by the other tokens in the input. In particular, the encoder may be configured such that the representations resolve the desired output of the transformer network.

In particular, the decoder may be configured so as to transform an input into a sequence of output tokens. In particular, the decoder may be configured so as to implement a masked self-attention mechanism such that each vector of a token is only influenced by the other tokens on one side of a sequence.

Furthermore, the decoder can be autoregressive, which means that intermediate results are fed back.

According to some examples, the input of the decoder can be based on the output of the encoder or be equivalent to the output of the encoder.

Moreover, the transformer network can comprise a classification module which is configured so as to map the output of the encoder to a set of learned outputs.

According to some examples, the training of a transformer model can take place in two phases, a pre-training phase and a fine-tuning phase. In the pre-training phase, a transformer model can be trained on a large body of data in order to learn the underlying semantics of the problem. Such pre-trained transformer models are available for different languages. For certain applications described here, fine-tuning may involve further training of the transformer network with medical texts with meanings annotated by experts and/or medical ontologies such as RADLEX and/or SNOMED. With the latter, the transformer model can learn typical relationships and synonyms of medical terms in particular, according to some examples.

One advantage of transformer networks is that they can efficiently handle long-range dependencies in input data due to the attention mechanism. Moreover, encoders used in transformer networks are capable of processing data in parallel, which saves computing resources during inference. Furthermore, due to autoregression, decoders of transformer networks are able to iteratively generate a sequence of output tokens with great confidence.

These details can be applied to the method for providing a trained function for determining a control sequence for deflecting an electron beam. The trained function can be implemented as one of the described models in order to determine an optimized control sequence based on the training input data, which comprises a map of dimensions of a focal spot. In particular, transformer networks or recurrent neural networks can be advantageous in order to model the temporal sequence of the deflection.

One or more example embodiments relates in a fourth aspect to an optimization system. The optimization system comprises an interface and a computing unit. The interface and the computing unit are designed so as to perform the above described method for determining a control sequence for deflecting an electron beam.

With regard to the definitions of features which are already comprised in the methods described above, reference is made to the description above.

The optimization unit can be designed as a technical system which is configured so as to solve an optimization task

The interface can be designed as a hardware or software interface, for example as a PCI bus, CAN bus, USB or Firewire. The interface can be designed so as to receive and/or transmit data.

The computing unit can comprise hardware and/or software components, for example a micro-processor or a so-called FPGA (field programmable gate array). The computing unit can be designed so as to perform calculations and process data.

The optimization system can be designed as a computer, micro-controller or integrated circuit.

Alternatively, the optimization system can be designed as a real or virtual computer network. A technical term for a real computer network is ‘cluster’, while a technical term for a virtual computer network is ‘cloud’. The optimization system can also be designed as a virtual system running on a computer, a real computer network or a virtual computer network. A technical term for this is ‘virtualization’.

The interface of the optimization system may in particular comprise a plurality of sub-interfaces that perform different method steps of the method. In other words, the interface may be designed as a plurality of interfaces.

The computing unit of the optimization system may in particular comprise a plurality of sub-computing units that perform different method steps of the method. In other words, the computing unit may be designed as a plurality of computing units.

The optimization system can be designed so as to receive a map of dimensions of a focal spot of an electron beam on an anode of an X-ray source for different positions of the focal spot on the anode. The map can be received by the interface. The different positions of the focal spot can be adjusted by deflecting the electron beam.

The optimization system can also be designed so as to determine a control sequence for deflecting the electron beam in dependence upon the map. The control sequence can be determined using the computing unit. The control sequence can be determined in such a manner that the effective focus shape is optimized with regard to the dimensions of the focal spot over an exposure period.

Finally, the optimization system can be designed so as to provide the specific control sequence. The control sequence can be provided using the interface.

The use of such an optimization system can offer multiple technical advantages. On the one hand, an improved image quality in the X-ray imaging can be achieved using the optimized control sequence. On the other hand, the efficiency of the X-ray source can be increased, since the focal spot dimensions are optimized over the entire exposure period. Furthermore, the optimization system can be adapted in a flexible manner to different X-ray sources and imaging requirements, which expands its versatility and range of applications.

One or more example embodiments relates in a fifth aspect to an X-ray source for use in a method described above for providing image data.

With regard to the definitions of features which are already comprised in the methods described above, reference is made to the description above.

The X-ray source is designed in particular so as to deflect the electron beam according to the control sequence.

In one embodiment of the X-ray source, the X-ray source can comprise an X-ray radiation unit. The X-ray radiation unit can comprise a cathode and an anode. The X-ray source can also comprise a deflection unit. The X-ray source can also comprise a single tank in which the X-ray radiation unit and the deflection unit can be arranged. The X-ray radiation unit can be enclosed by a vacuum housing. The deflection unit can be designed so as to deflect the electron beam in the X-ray radiation unit according to the control sequence.

A deflection unit can be designed as an apparatus which is configured so as to deflect an electron beam. The deflection unit can, for example, generate electric, magnetic or electro-magnetic fields in order to influence the electron beam. Possible embodiments of a deflection unit include electromagnets, electrostatic deflection plates or coils.

The deflection unit can be designed so as to deflect the electron beam in the X-ray unit according to the control sequence. This means that the deflection unit can control and move the electron beam corresponding to the predetermined pattern or instructions which are defined in the control sequence. The control sequence can contain, for example, information regarding the direction, strength and temporal sequence of the deflection.

A single tank can be designed as a housing in which the X-ray radiation unit and the deflection unit are arranged. The single tank can be designed so as to protect and shield the components of the X-ray source. The single tank can be manufactured from materials such as metal or synthetic material. The single tank can be filled with an insulating medium, such as for example oil, in order to ensure an electrical insulation.

A vacuum housing can be designed as a closed chamber in which a vacuum or a very low pressure is prevailing. The vacuum housing can enclose the X-ray radiation unit in order to ensure optimum conditions for the generation and propagation of the electron beam. The vacuum housing can be manufactured from materials, such as glass, ceramic or metal.

The deflection unit is arranged in this case within the single tank but outside the vacuum housing.

The X-ray source can optionally comprise a focusing unit within the X-ray radiation unit. In particular, the focusing unit can be part of a focusing head of the X-ray source. The focusing unit can be designed as a component of the X-ray source and is used to concentrate and focus the electron beam. The focusing unit can comprise electro-magnetic or electro-static lenses which form the electric or magnetic field in such a manner that the electron beam is concentrated on a small as possible focal spot on the anode. The strength of the focusing unit can be varied in order to render possible optimum focusing for different operating parameters. In some embodiments, the focusing unit can also be used to influence the shape of the focal spot in order to fulfill specific imaging requirements. In cooperation with the deflection unit, the focusing unit can contribute to the optimization of the effective focus shape over the entire exposure period.

The described arrangement of the components in a single tank with the vacuum housing can render possible a compact and robust construction of the X-ray source. By integrating the deflection unit into this arrangement, it is possible to achieve a precise control of the electron beam, which can lead to an improved image quality and reduced thermal load on the anode. In addition, an arrangement of the deflection unit outside the vacuum housing offers the advantage of a protection against electrical flashovers between the components.

In a further embodiment of the X-ray source, the deflection unit may comprise an air coil.

The air coil can be designed as a coil which does not have a ferro-magnetic core. The air coil can be produced from an electrically conductive material, such as for example copper or aluminum. The air coil can have a cylindrical or prismatic shape and consist of multiple windings of the conductive material. The coil can be designed as a Helmholtz coil. The precise geometry and number of windings of the air coil can be configured corresponding to a desired deflection characteristic of the electron beam.

The air coil can be designed so as to generate a magnetic field when an electric current flows through the windings. This magnetic field can be used to deflect the electron beam in the X-ray radiation unit. In comparison to coils with a ferro-magnetic core, an air coil generally has a lower inductance, which renders possible more rapid changes in the magnetic field. In addition, the ferro-magnetic core does not require any installation space.

The use of an air coil as part of the deflection unit can offer different advantages. On the one hand, an air coil renders possible a precise and rapid control of the electron beam, since the generated magnetic field is directly proportional to the applied current and there are no hysteresis effects. On the other hand, an air coil can be more compact and lighter in comparison to coils with a ferro-magnetic core, which can be of advantage in the confined environment of an X-ray source.

Furthermore, an air coil can have a high degree of linearity between the applied current and the generated magnetic field, which renders it possible to control the electron beam deflection in a precise manner according to the control sequence. The air coil can also function efficiently at high frequencies, which can be advantageous for rapid deflection movements of the electron beam.

In an alternative embodiment of the X-ray source, the X-ray source can comprise an X-ray radiation unit which comprises a cathode, an anode and a deflection unit. The X-ray source can also comprise a single tank in which the X-ray radiation unit can be arranged. The X-ray radiation unit can be enclosed by a vacuum housing. The deflection unit can be designed so as to deflect the electron beam in the X-ray radiation unit according to the control sequence.

Reference is made to the description above with regard to the definition of the single tank, the vacuum housing and the deflection unit.

In this exemplary embodiment, the deflection unit is arranged within the vacuum housing.

By arranging the deflection unit within the vacuum housing of the X-ray radiation unit, a compact construction is possible and additional vacuum feedthroughs are avoided.

In a further embodiment of the X-ray source, the deflection unit can deflect the electron beam via electric or magnetic deflection.

The electric deflection takes place via electric fields. Deflection plates can be used for this purpose, for example, between which an electric field is generated. The electron beam can be deflected when passing through this electric field. The strength and direction of the deflection can be controlled by the applied voltage at the deflection plates. In particular, the focusing unit can be at least part of the deflection unit. The focusing unit in this case can be comprised by a focusing head of the X-ray source.

The magnetic deflection can take place via magnetic fields. For example, coils which generate a magnetic field can be used for this purpose. The electron beam can be deflected when passing through this magnetic field. The strength and direction of the deflection can be controlled by the current in the coils. In this case, the coil can be an air coil, for example.

By using an electric or magnetic deflection, the electron beam can be deflected in a precise and rapid manner without the need for mechanically movable parts. This can contribute to an improved reliability and serviceable life of the X-ray source. In addition, it is possible to achieve an optimized movement of the focal spot on the anode as a result of the rapid deflection and this can lead to an improved image quality.

In a further optional, alternative embodiment, the X-ray source can comprise multiple emitters. In this case, the movement of the focal spot can be implemented by activating in a targeted manner the emitter in a specific sequence. The emitters can be designed as separate cathodes which are arranged in a defined pattern on a carrier. Alternatively a cathode can comprise multiple emitters. Sequential or overlapping activation of the emitters can generate effective movement of the focal spot on the anode without requiring physical deflection of the electron beam. This arrangement can render possible an extremely precise control of the focal spot position and shape, since the activation times and intensities of the individual emitters can be controlled individually. In addition, this method can render possible a faster focal spot movement than mechanical deflection systems or electro-magnetic deflection systems when using thermionic emitters (filaments). A further advantage may exist in the increased redundancy and serviceable life of the system, since if one emitter fails, the others can continue to be used.

One or more example embodiments relates in a sixth aspect to an imaging system. The imaging system comprises an interface, a computing unit, an X-ray source and an X-ray detector. The interface, the computing unit, the X-ray source and the X-ray detector can be designed so as to perform the method for providing image data.

With regard to the definitions of features which are already comprised in the methods and/or systems described above, reference is made to the description above.

The imaging system can be designed as a system which is configured so as to generate and provide image data.

The interface and the computing unit can be designed in a manner analogous as described above with regard to the optimization system in general. The X-ray source can be designed according to the description above.

The X-ray detector may be designed as an apparatus designed so as to detect the X-ray radiation generated by the X-ray source and transmitted through an examination object. The X-ray detector can be designed, for example, as a planar detector or a line detector.

By combining these components in one imaging system, it is possible to generate medical image data in an efficient and precise manner. The deflection of the electron beam for moving the focal spot can contribute in this case to optimizing the effective focus shape and thus to an improved image quality.

In one embodiment of the imaging system, the imaging system can be designed as a mammography system. The mammography system can also comprise a compression unit for compressing an examination object. The compression unit can be arranged between the X-ray source and the X-ray detector.

The mammography system can be defined as a specialized medical imaging system which is configured to examine and image a human breast. The mammography system renders it possible to create high-resolution X-ray images of breast tissue from the breast for the early detection of breast cancer and other abnormalities.

The mammography system can additionally comprise a compression unit. A compression unit can be defined as an apparatus which is designed so as to compress an examination object, in this case the breast. By compressing the breast, it is possible to reduce the thickness of the tissue to be irradiated and this can lead to an improved image quality and a lower radiation dose.

In the context of a mammography system, the examination object is typically a human breast. However, the examination object may also be a phantom or other test object used for calibration or quality assurance purposes.

The compression unit can comprise two plates or compression plates-a movable upper compression plate and a fixed lower compression plate. The compression plates can be arranged in such a manner as to be able to move relative to one another. In order to compress the examination object, the first compression plate can be moved relative to the second compression plate, so that the examination object is compressed between the first compression plate and the second compression plate. The lower compression plate can be used in particular as a support surface for the breast, whereas the upper compression plate can be lowered in order to compress the breast. In particular, the lower compression plate can be designed as a cover of the X-ray detector. Alternatively, both compression plates can be movable.

The compression plates can be manufactured from different materials, which are transparent for X-ray radiation, such as for example carbon fiber or special synthetic materials. This renders it possible for X-ray radiation to pass through the compression plates with minimal attenuation.

The compression unit can be designed so as to compress the examination object with a defined pressure or a defined force. The defined force can be between 10 N and 200 N, for example. The compression unit can comprise a pressure measuring unit or a force measuring unit which is designed so as to measure the pressure or force exerted on the examination object.

The compression unit can be actuated manually or in an automated manner. In the case of a manual actuation, the compression unit can be actuated by an operator of the mammography system. In the case of an automated actuation, the compression unit can be controlled by a control device of the mammography system.

The design of the imaging system as a mammography system with a compression unit can offer the advantage of enabling precise and gentle examination of the breast. The compression of the breast can lead to an improved visualization of micro-calcifications and other tissue changes, which can support early recognition of breast cancer. The above described method can perform in particular three-dimensional imaging such as tomosynthesis but also a two-dimensional mammogram quicker and/or with an improved image quality.

In a further embodiment of the imaging system, the X-ray source can be designed so as to be able to pivot relative to the X-ray detector and/or the compression unit in order to perform a planned movement.

The pivotable design of the X-ray source can be achieved by different technical implementations. For example, the X-ray source can be attached to a pivot arm which can rotate about an axis. Alternatively, the X-ray source can be mounted on a curved rail, along which it can be moved.

Optionally, the X-ray source can comprise at least two collimator plates which may be designed to shape and limit the X-ray beam. Alternatively, the at least two collimator plates can be designed as part of a collimator as a separate component. In particular, the at least two collimator plates can be arranged on the X-ray source, in particular at an exit window or radiation exit window of the X-ray source. In particular, the at least two collimator plates can then be flanged to a single tank of the X-ray source. The collimator plates can be manufactured from a radiation-absorbing material such as steel, gold, lead or tungsten and can be arranged in front of an exit window of the X-ray radiation from the X-ray source. The positioning of the collimator plates in front of the exit window renders it possible for the exiting X-ray radiation to be controlled in a precise manner. Optionally, the collimator plates can be moved together with the planned movement of the X-ray source. This movement can ensure that the same region of the surface of the X-ray detector is always exposed to the emitted X-ray radiation. As a result, it is possible to achieve a uniform exposure of the detector over the entire imaging period, which can contribute to an improved image quality.

The collimator plates can additionally or alternatively to the movement of the X-ray source be moved together with the movement of the focal spot. This co-movement of the collimator plates can ensure that for each position of the focal spot the same region of the surface of the X-ray detector is exposed to the emitted X-ray radiation. The synchronization of the movement of the collimator plates with the focal spot movement renders in possible to control the X-ray beam in a precise manner, irrespective of the actual position of the focal spot on the anode. This can lead to a uniform exposure of the detector over the entire imaging period and thus improve the image quality. The co-movement of the collimator plates can be achieved by a separate mechanism or by a coupling to the deflection system of the electron beam.

The combination of the pivotable design of the X-ray source with the deflection of the electron beam according to a control sequence can offer additional advantages. For example, the movement of the focal spot by the deflection of the electron beam can be combined with the movement of the X-ray source in order to further optimize the effective focus shape. Pivoting the X-ray source renders it possible that the X-ray source assumes different positions and angles during imaging, comprising multiple exposure periods. This can render it possible to record project images of the examination object from different angles, which can be advantageous for specific imaging methods, such as tomosynthesis.

One or more example embodiments relates in a seventh aspect to a training system. The training system comprises a training interface and a training computing unit. The training interface and the training computing unit are designed so as to perform the method for providing a trained function for determining a control sequence deflecting an electron beam.

In this case, the training computing unit and the training interface can be designed in a manner analogous to the interface and computing unit described above with regard to the optimization system.

The training system can also comprise a training storage unit. The training storage unit can be designed as a non-permanent working memory (RAM) or as a permanent mass storage device (hard drive, USB stick, SD card, solid state disk). The training storage unit can be designed to store the training input data, the training output data and the trained function.

One advantage of the training system can be that it renders possible to provide a trained function in an efficient and automated manner. It is possible by using specialized hardware and software components to accelerate and optimize the training of the function. The modular structure with a training structure and training computing unit also renders it possible to adapt in a flexible manner to different training tasks and data formats.

One or more example embodiments relates in an eighth aspect to a computer program product with a computer program that can be loaded directly into a storage device of an imaging system. The computer program product comprises program segments for performing all of the above-described steps of the method for providing image data when the program segments are executed by the imaging system.

The computer program product can be designed as a software packet or a software application that can be installed and executed on a computer system. The computer program product can comprise program codes or program segments which contain instructions for a computer in order to perform the specific functions.

The computer program can be loaded directly into a storage device of an imaging system. This means that the computer program can be configured in such a manner that it can be loaded directly from the storage medium into the working memory of the imaging system and can be executed there without requiring additional installation.

The storage device of the imaging system can be designed as a data storage device which is used to store data and programs for processing by the imaging system. The storage device may be configured, for example, as a hard disk storage device, a solid-state drive or a random access memory (RAM).

The imaging system may be configured as described above as a technical system used to generate images. In the medical context, an imaging system can comprise, for example, an X-ray system, a mammography system or a computed tomography system.

Program segments can be designed as parts or modules of a computer program which performs specific functions or tasks. The program segments can be written in a programming language and contain instructions for the computer.

The execution of program segments by the imaging system may mean that the processor or computing unit of the imaging system processes the instructions in the program segments and performs the corresponding operations.

The program segments of the computer program product may be designed so as to perform the steps of the above-described method for providing image data when performed by the imaging system.

The computer program product can be designed as a collection of computer program instructions stored on a computer-readable medium. The computer-readable medium can be, for example, a hard drive, a USB stick, an SD card or a solid state disk.

The computer program can be written in a programming language such as C++, Java or Python. The program segments can comprise functions, methods or routines that implement the individual steps of the claimed method.

The storage device of the imaging system into which the computer program can be loaded can be designed as a volatile working memory such as RAM or as non-volatile memory such as a hard disk.

The imaging system can be designed so as to execute the loaded computer program and thereby perform the steps of the claimed method. This may include, for example, generating X-ray radiation, detecting X-ray radiation and providing image data.

The program segments can be executed by the imaging system using a processor or a computing unit of the imaging system. In this case, the individual method steps can be performed sequentially or in parallel.

By using a computer program product, the claimed method can be implemented in a flexible manner on different imaging systems. This renders it possible to easily update and customize the process without changes to the imaging system hardware.

One or more example embodiments relates in a ninth aspect to a computer-readable storage medium. The computer-readable storage medium can store program segments that are readable and executable by an imaging system. The program segments can be designed so as to perform all the steps of the above-described method for providing image data when the program segments are executed by the imaging system.

The computer-readable storage medium can be designed as a physical medium that is configured so as to be readable and executable by an imaging system and to store executable program segments. The computer-readable storage medium can be, for example, a hard drive, a USB stick, an SD card or a solid state disk (SSD).

The program segments can be designed as parts of a computer program which performs specific functions or tasks. The program segments can be written in a programming language such as C++, Java or Python.

The imaging system can be designed as described above as a system which is configured so as to generate and process medical images. The imaging system can be designed, for example, as an X-ray system or a computed tomography system.

The program segments can be designed as readable when they are in a format which can be interpreted by the imaging system. The program segments can be designed as executable when they can be directly executed by the imaging system without requiring further compilation or interpretation.

Performing the program segments can be designed as a process in which the imaging system processes the instructions contained in the program segments. This can include performing calculating, controlling hardware components or processing data.

The steps of the method can be designed as a sequence of operations which are performed by the imaging system in order to achieve a specific result. These steps can include, for example, detecting X-ray radiation, processing image data or controlling the movement of an X-ray source.

The program segments can be executed by the imaging system using a computing unit of the imaging system. The computing unit can, for example, be designed as a micro-processor, an FPGA (field programmable gate array) or another suitable computing unit.

The computer-readable storage medium with the program segments stored on it can render it possible for the imaging system to perform the method according to one or more example embodiments for providing image data without requiring additional hardware modifications to the imaging system. This renders it possible to implement one or more example embodiments in existing imaging systems in a flexible and cost-effective manner.

Advantageously, the use of a computer-readable storage medium with the program segments stored thereon renders possible an easy distribution and installation of the method according to one or more example embodiments on various imaging systems. This renders possible quick and uncomplicated updating of existing imaging systems with the new functionality.

One or more example embodiments relates in a tenth aspect to a computer program product with a computer program that can be loaded directly into a storage device of an optimization system. The computer program comprises program segments for executing all of the steps of the method for providing a control sequence when the program segments are executed by the optimization system.

The computer program product can be designed in a manner analogous to the general description of the computer program product described above with regard to the method for providing image data. This general description can be transferred in a manner analogous to the computer program product for execution on the optimization system.

By executing the program segments on the optimization system, it is possible in an efficient and automated manner to determine an optimized control sequence for deflecting an electron beam. This can lead to improved image quality while reducing radiation exposure for the patient.

One or more example embodiments relates in an eleventh aspect to a computer-readable storage medium. Readable and executable program segments can be stored on the computer-readable storage medium by an optimization system. The program segments can be designed so as to perform all the steps of the method for providing a control sequence. The program segments can be executed by the optimization system.

The computer-readable storage medium can be designed in a manner analogous to the description of the computer-readable storage medium with regard to the above-described method for providing image data. Execution on the imaging system can be transferred analogously to execution on the optimization system.

By storing the program segments on a computer-readable storage medium, the method can be flexibly performed on different optimization systems. This renders it possible to easily distribute and update easy distribution and updating of the software for performing the method.

FIG. 1 shows an exemplary embodiment of a method for providing image data.

The method comprises a method step of generating GEN X-ray radiation R via an X-ray source 400. In this case, the X-ray source 400 comprises a cathode 411 for generating an electron beam and an anode 412. The electron beam is accelerated onto the anode. In this case, the electron beam striking the anode 412 forms a focal spot 430. X-ray radiation is generated by decelerating the electron beam in the focal spot 430 and exciting the anode material. In this case, the focal spot 430 is moved on the anode 412 during an exposure period AT by deflecting the electron beam on the anode 412. In this case, the deflection is based on a predetermined control sequence, so that the movement of the focal spot 430 is not continuous in sections.

The method comprises a further method step of detecting REC X-ray radiation R via an X-ray detector 505 during the exposure period AT. In this case, at least a portion of the X-ray radiation R emitted from the X-ray source 400 is detected by the X-ray detector.

The method comprises a further method step of providing PROV the image data based on the detected X-ray radiation R.

In one exemplary embodiment, the control sequence can comprise discrete deflection values. The deflection values can indicate in this case input voltage values or current values or explicit angles with which the electron beam can be deflected using a deflection unit. Alternatively or additionally, the deflection values can indicate specific positions of the focal spot 430 on the anode 412.

The control sequence can specify in this case in particular a temporal progression of the deflection values comprised by the control sequence.

In one exemplary embodiment, the control sequence can specify a type and/or a speed of the deflection.

The type of deflection can specify in particular how the electron beam is to be moved between two deflection values. For example, the electron beam can jump between two deflection values. In other words, the electron beam can jump from one deflection to the next according to the control sequence or the deflection values. Alternatively, the electron beam can be moved continuously between two deflections or deflection values.

The speed of the deflection of the electron beam indicates how quickly the electron beam is to be moved between two deflection values. In other words, the speed indicates how quickly the deflection or the deflection angle of the electron beam is to be varied.

In one exemplary embodiment, the control sequence comprises two maximum deflection values. These define in each case a maximum deflection of the electron beam on the anode 412. The maximum deflections can be designed in this case opposite to one another from the perspective of the zero position.

In one exemplary embodiment, the control sequence comprises in addition to the two maximum deflection values at least a further deflection value. The further deflection value is arranged between the maximum deflection values. In particular, the further deflection value is arranged spatially on the anode between the two maximum deflection values. Alternatively or additionally, the further deflection value can be arranged according to the control sequence temporally between the two maximum deflection values. In other words, the control sequence is designed in such a manner that the further deflection value is adjusted or the electron beam is deflected accordingly after the first maximum deflection value but before the second maximum deflection value.

In one exemplary embodiment, the X-ray source 400 can be moved during the exposure period AT according to a planned movement. The planned movement can be in particular radial or curved or linear. In particular, the planned movement can be designed to perform tomosynthesis. In particular, the X-ray source 400 can be pivoted relative to the X-ray detector 505.

In one exemplary embodiment, the speed of the movement of the focal spot 430 generated by the deflection is different at least during a part of the exposure period AT to a speed of the planned movement of the X-ray source 400. In this case, the inequality refers in particular to an amount of speed.

In one exemplary embodiment, the movement of the focal spot 430 generated by the deflection of the electron beam takes place at least partially in the direction of the planned movement of the X-ray source 400.

According to one exemplary embodiment, the control sequence comprises at least four different deflection values. In this case, the control sequence comprises each of the deflection values at least twice. In this case, the electron beam can be deflected in such a manner that it is moved continuously between at least two deflection values following one another according to the control sequence opposite the direction of the movement of the X-ray source. In this case, the electron beam is deflected in such a manner that it jumps between at least two further deflection values following one another according to the control sequence in the direction of the movement of the X-ray source.

According to a further exemplary embodiment, the above described method is repeated or performed multiple times during the planned movement of the X-ray source 400. In other words, at least two image data items are acquired and provided.

According to a further exemplary embodiment, the movement of the X-ray source is continuous.

According to one exemplary embodiment, the control sequence comprises at least two consecutive discrete deflection values. In this case, the electron beam is deflected in such a manner that it jumps between at least two discrete deflection values following one another according to the control sequence.

According to one exemplary embodiment, the electron beam is deflected in such a manner that the movement of the focal spot is partially continuous.

According to one exemplary embodiment, the method comprises an optional method step of focusing FOC the electron beam around a zero position of the focal spot 430 via a focusing unit. In this case, the zero position is in particular the position of the focal spot 430 on the anode 412 which the electron beam strikes when not deflected. The focusing unit can be in particular part of the focusing head 415 or can be comprised by the focusing head 415. It is possible in particular to vary the strength of the focusing. In particular, the focusing can be of a magnitude of an applied acceleration voltage between the cathode 411 and the anode 412.

According to one embodiment, the further the focal spot is moved away from the zero position by deflecting the electron beam, the lower the focusing using the focusing unit. In other words, with a greater distance between the focal spot 430 and the zero position, the strength of the focusing in the focusing step FOC can be reduced. In particular, there can be a linear or non-linear relationship between the distance of the focal spot 430 from the zero position and the strength of the focusing.

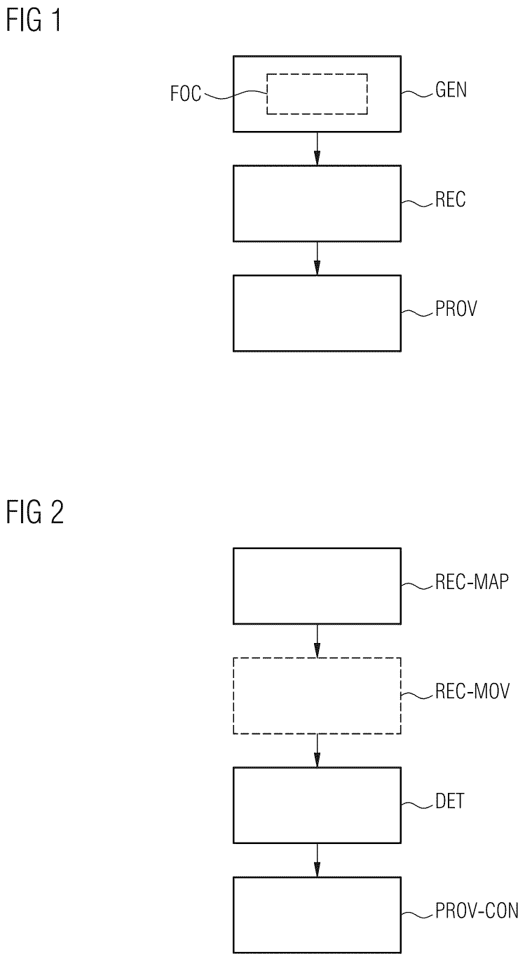

FIG. 2 shows an exemplary embodiment of a method for providing a control sequence.

The method comprises a method step of receiving REC-MAP a map of dimensions DIM of a focal spot 430. In this case, the focal spot 430 is formed by an electron beam striking an anode 412 of an X-ray source 400. In this case, the dimensions DIM depend upon a deflection of the electron beam.

In particular, the dimensions DIM can additionally depend on an acceleration voltage applied between the cathode 411 and the anode 412 for accelerating the electrons onto the anode 412. In particular, the dimensions DIM can additionally depend on a tube current which is applied to the cathode 411 or to the emitter of the cathode 411.

The method also comprises a further method step of determining DET a control sequence for deflecting the electron beam in dependence upon the map. In this case, the control sequence is determined in such a manner that an effective focus shape of X-ray radiation emitted by the focal spot 430 is optimized over an exposure period AT with regard to the dimensions DIM of the focal spot 430.

The method comprises a further method step of providing PROV-CON the control sequence.

According to one exemplary embodiment, the control sequence provided is designed for use in a method described in FIG. 1.

According to one exemplary embodiment, the method comprises an optional method step of receiving REC-MOV a planned movement of the X-ray source 400 during the exposure period AT. In this case, the planned movement of the X-ray source 400 is taken into account when determining DET the control sequence.

According to one exemplary embodiment, the method step of determining DET the control sequence can comprise a method step of applying a trained function to the map and optionally the planned movement of the X-ray source 400. The control sequence is determined by applying the trained function. In other words, the object or the result of the trained function is the control sequence.

According to an alternative exemplary embodiment, the method step of determining DET the control sequence can comprise a method step of adjusting an optimizing function in dependence upon the map and optionally the planned movement of the X-ray source 400, and a method step of determining the control sequence based on the optimization function.

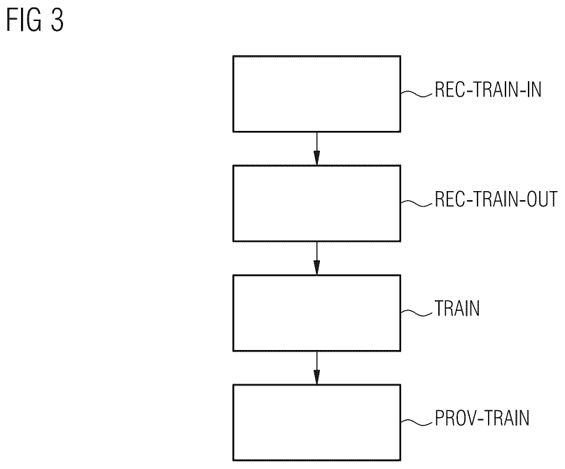

FIG. 3 shows an exemplary embodiment of a method for providing a trained function.

In this case, the trained function is trained so as to determine a control sequence for deflecting an electron beam. The trained function is designed for use in a method described according to FIG. 2.

The method comprises a method step of receiving REC-TRAIN-IN training input data. In this case, the training input data comprises a map of dimensions DIM of a focal spot 430. In this case, the focal spot 430 is formed by an electron beam striking an anode 412 of an X-ray source 400. In this case, the dimensions DIM depend at least upon a deflection of the electron beam. In this case, the training input data also optionally comprises a planned movement of the X-ray source 400.

The method comprises a further method step of receiving REC-TRAIN-OUT training output data. In this case, the training output data comprises control sequences with which optimized effective focus shapes of X-ray radiation R emitted by the focal spot 430 can be achieved.

The method also comprises a further step of training TRAIN the trained function based on the training input data and the training output data.

The method also comprises a method step of providing PROV-TRAIN the trained function.

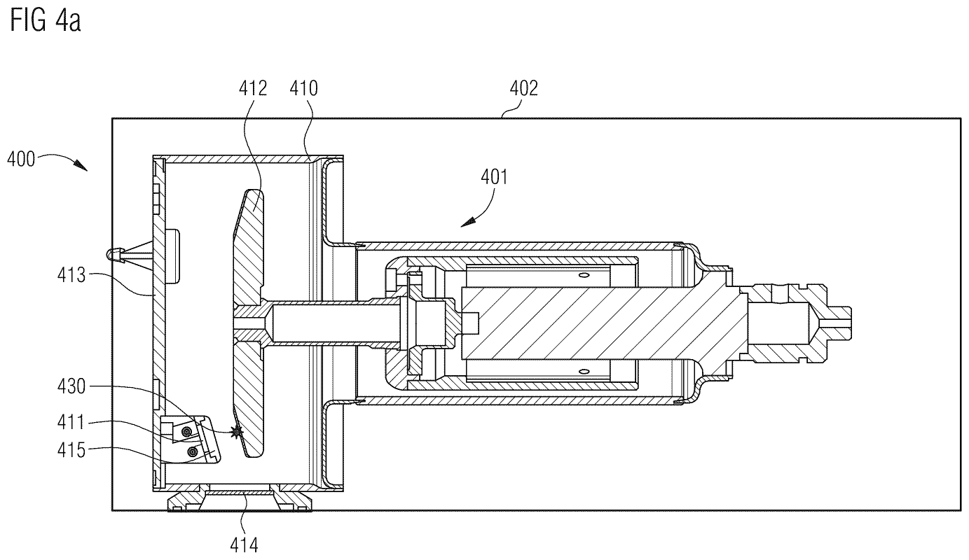

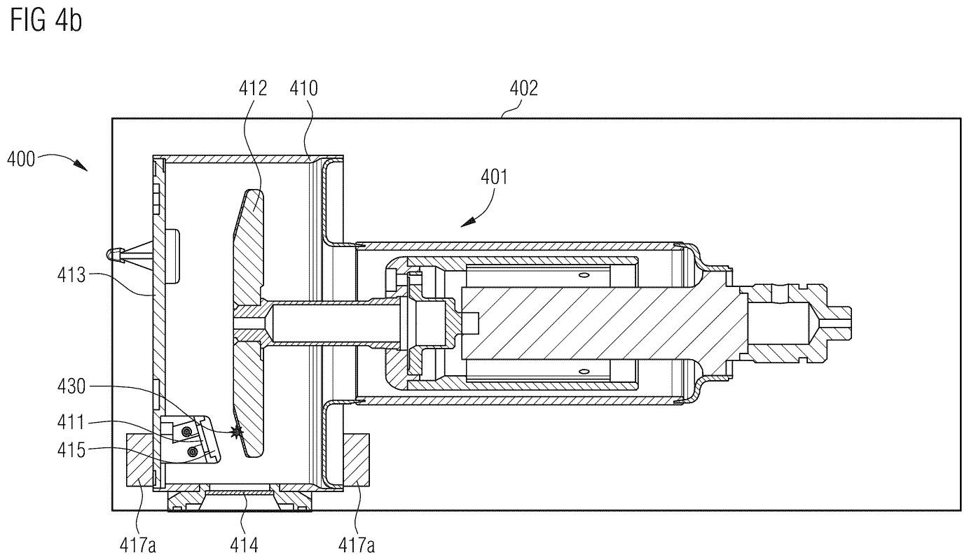

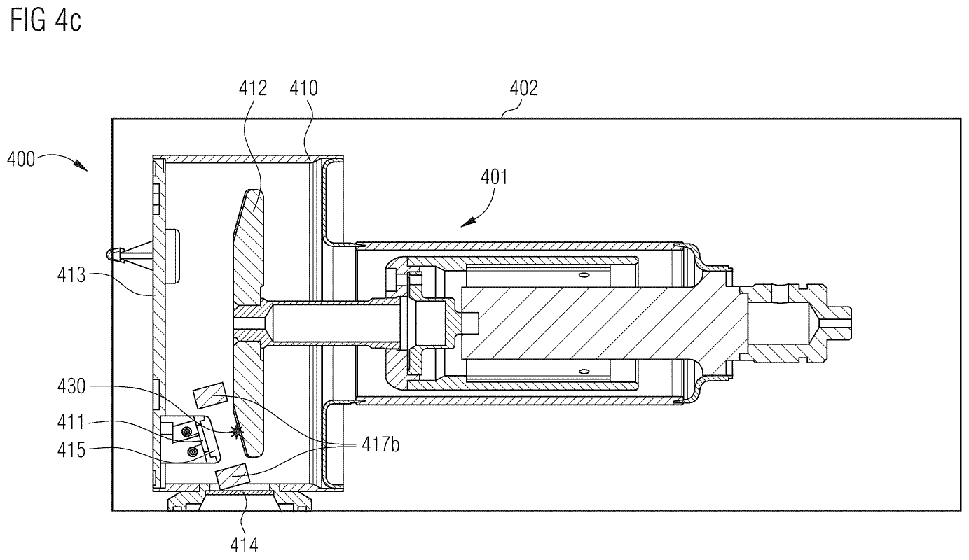

FIG. 4a shows an exemplary embodiment of an X-ray source 400. The X-ray source 400 comprises an X-ray radiation unit 401. The X-ray radiation unit 401 is enclosed by a vacuum housing 410. The X-ray radiation unit 401 comprises a cathode 411 and an anode 412. The cathode 411 and the anode 412 are arranged within the vacuum housing 410. The vacuum housing 410 can be sealed in a vacuum-tight manner by a cathode cover 413. The cathode 411 emits electrons which are accelerated at least partially in the direction of the anode 412 and when striking a focal spot 430 generated X-ray radiation in the material of the anode 412. The accelerated electrons can be referred to as an electron beam or an electron cloud. The anode 412 is configured as a rotating anode so that the focal spot 430 forms a focal path. The generated X-ray radiation R exits the vacuum housing 410 as a bundle of rays via an exit window or a beam exit window. The X-ray radiation R can exit an beam exit window 414 of the X-ray source 400 through an opening which can be covered by a window or a filter in order to block or reduce undesired radiation.

The anode 412 designed as a rotating anode can be designed as a rotationally symmetric body which can be coupled or is coupled to an anode tube and a heat storage device and has a focal path for generating X-ray radiation. The rotationally symmetric body is in particular an X-ray radiation anode cup or an X-ray radiation anode plate. Typically, the body contains molybdenum and/or consists of molybdenum or a molybdenum-based alloy (for example TZM or MHC). Typically, the focal path has tungsten and/or consists of tungsten or a tungsten-based alloy (for example tungsten-rhenium). The body is in particular a carrier body for the focal path. The focal path can be different structurally to the carrier body, in particular in terms of material. The anode 412 is in particular suitable for a medical or non-medical imaging in a kV range between 10 and 200 kV. The kV range describes in particular a maximum acceleration voltage which specifies a maximum photon energy of the X-ray radiation which can be generated. The X-ray radiation is emitted from the upper side of the anode which has in particular a suitable shape and suitable material in order to achieve a high X-ray radiation yield.

The anode 412 designed as a rotating anode is fundamentally suitable to be rotatably mounted about a rotational axis. In order to be rotatably mounted, the axis of rotation is typically coupled to the anode tube. The anode tube may be hollow or made of solid material in another design. The anode tube can be coupled to a rotor of a rotary drive. The anode tube is typically designed rotationally symmetrical about the axis of symmetry of the body.

In one embodiment, the anode 412 has a heat storage device, wherein the lower side of the body is coupled in a peripheral section in a heat-conducting manner to the heat storage device, wherein the heat storage device has a higher thermal conductivity and/or a higher heat capacity than the body. The heat storage device is typically made from graphite. The heat storage device is typically coupled to the lower side of the body via a fastening means, for example soldering, in particular graphite soldering (for example, titanium or zirconium soldering). The heat storage device is typically designed rotationally symmetrical about the axis of symmetry of the body. The heat-conducting coupling of the lower side of the body to a heat storage device which has a higher heat conducting capacity and/or heat capacity renders it possible to distribute and store heat effectively, which can mitigate temperature peaks during operation. By using a heat storage device with superior thermal properties, it is possible to increase the thermal stability of the appliance, which can lead to a more reliable function under different operating conditions. The improved heat dissipation and storage can reduce the necessity for additional cooling, which increases the energy efficiency of the appliance and reduces operating costs.

The cathode 411 can comprise in particular an electron emitter and/or a focusing head 415. The electron emitter can be in particular a cold electron emitter or a warm electron emitter. The electron emitter is, for example, a hot cathode or a field emission cathode. The hot cathode generates electrons by thermal emission in which the electrons are released from the material by heating. This is referred to as hot electron emission or thermionic emission. The field emission cathode generates electrons through the field effect in which the electrons are drawn by a strong electric field out of the material present in the peaks. This is referred to as cold electron emission. The cathode 411 is preferably switched to a negative voltage in contrast to the anode 412 in order to accelerate the electrons. The acceleration voltage determines the energy and the intensity of the generated X-ray radiation R.

The vacuum housing 410 can be evacuated or is evacuated, in particular with a high vacuum. The vacuum housing 410 is made of metal and/or glass, for example. The acceleration voltage can preferably be applied between the cathode 411 and the anode 412. In particular, a high voltage generator can provide the acceleration voltage.

The X-ray radiation unit 401 is arranged above bearings in a single tank 402. The single tank 402 can be filled with an oil for the thermal and/or electrical insulation. The single tank 402 and the bearings, the electronic system likewise arranged in the single tank 402, the cooling system, etc. are not shown for reasons of clarity. It is known from the prior art how such standard components can be arranged and designed.

FIG. 4b shows a first exemplary embodiment of an X-ray source 400 with a deflection unit 417a. The X-ray source 400 is fundamentally constructed according to the description relating to FIG. 4a.

The deflection unit 417a is arranged in this case outside the vacuum housing 410 but within the single tank 402. Alternatively, the deflection unit 417a can also be arranged outside the single tank 402. The deflection unit 417a can be arranged in this case in particular about the radiation exit window 414.

The deflection unit 417a can be designed in one exemplary embodiment as a coil, in particular as an air coil.

Alternatively, the deflection unit 417a can be designed as an electro-static deflection unit.

FIG. 4c shows a second exemplary embodiment of an X-ray source 400 with a deflection unit 417b. The X-ray source 400 is fundamentally constructed according to the description relating to FIG. 4a.

The deflection unit 417b is arranged in this case within the vacuum housing 410.

In one exemplary embodiment, the deflection unit 417b can be part of the focusing head 415.

The deflection unit 417b can deflect the electron beam via electric or magnetic deflection. The deflection unit 417b can be designed for this purpose as a coil and/or deflection plates.

In embodiments of the invention, a deflection unit 417a outside the vacuum housing 410 can be combined as described in FIG. 4b with a deflection unit 417b in the vacuum housing 410 as described in FIG. 4c.

FIG. 5 shows an exemplary embodiment of an imaging system. The imaging system is designed in this case in particular as a mammography system 500.

The mammography system 500 is shown in an exemplary and roughly schematic representation. The mammography system 500 can be designed in particular for tomosynthesis. Relative directions such as ‘up’, ‘down’, etc. refer to the mammography system 500 which has been set up for its intended use. The mammography system 500 has a vertical column 507 and a source-detector arrangement 503, which in turn comprise an X-ray source 400 and an X-ray detector 505 with a cover 505.1. The vertical column 507 is mounted on the ground. The source-detector arrangement 503 can be connected so as to be able to move to the vertical column 507, so that the height of the cover 505.1 of the X-ray detector 505 can be adjusted to a breast height of a patient.

A breast O of the patient (shown here schematically) is the examination object and in embodiments is positioned for examination on the cover 505.1 of the X-ray detector 505. The cover 505.1 forms thereby a second compression plate. A compression plate 506 which is connected so as to be able to move or be moved to the source-detector arrangement 503 is arranged over the breast O and the cover 505.1. For the purposes of the examination, the breast O is compressed and simultaneously fixed, by lowering the compression plate 506 onto it, so that pressure is exerted on the breast O between the compression plate 506 and the cover 505.1. Alternatively, it is possible to use a second compression plate for the compression instead of the cover 505.1 of the X-ray detector 505, so that the breast O is compressed between two compression plates. The two compression plates or the compression plate 506 and the cover 505.1 of the X-ray detector 505 are comprised by a compression unit 501. In addition, the compression unit 501 can also comprise a positioning unit in order to move the compression plate 506 or the compression plates for compression purposes.

The X-ray source 400 is arranged with respect to the X-ray detector 505 in such a manner that the X-ray detector 505 detects X-ray radiation R emitted from the X-ray source 400 after at least a part of the X-ray radiation R can passed through the breast O.

In embodiments, the X-ray source 400 can be movable or rotatable or pivotable relative to the X-ray detector 505 via a rotating arm 508, for example, in a range of ±25° about a basic position in which the rotating arm 508 is perpendicular to the cover 505.1 of the X-ray detector 505.

The mammography system 500 can comprise in particular a control device 510 with an interface 510.1, a computing unit 510.2 and a storage device unit 510.3. The control device 510 can be connected in particular to a terminal 513 which has, for example, a user interface or display unit via which a user can transmit commands to the mammography system 500 or call up measurement results, for example the recorded projection images or X-ray images. The control device 510 can be located in the same room as the mammography system 500, however, it can also be located in an adjacent control room or at an even greater spatial distance.

The control device 510 is designed in this case so as to perform a method according to one or more example embodiments for determining a three-dimensional shape of the breast. The control device 510 comprises an interface 510.1, a computing unit 510.2 and a storage device unit 510.3.

The control device 510 can be in particular a computer, a micro-controller or an integrated circuit (IC). Alternatively, the control device 510 may be a real or virtual computer network (a technical term for a real computer network is ‘cluster’, a technical term for a virtual computer network is ‘cloud’). The control device 510 can be designed as a virtual system running on a computer or a real computer network or a virtual computer network, (a technical term for this is ‘virtualization’).

The interface 510.1 can be a hardware or software interface, (for example as a PCI bus, CAN bus, USB or Firewire). The computing unit 510.2 can comprise hardware and/or software components, for example a micro-processor or a so-called FPGA (field programmable gate array). The storage device unit 510.3 can be designed as a non-permanent working memory (random access memory, RAM) or as a permanent mass storage device (hard drive, USB stick, SD card, solid state disk (SSD)).

The interface 510.1 can in particular comprise a plurality of sub-interfaces that perform different method steps of the respective method according to one or more example embodiments. In other words, the interface 510.1 can be designed as a plurality of interfaces 510.1. The computing unit 510.2 can in particular comprise a plurality of sub-computing units that perform different method steps of the respective method according to one or more example embodiments. In other words, the computing unit 510.2 can be designed as a plurality of computing units 510.2.

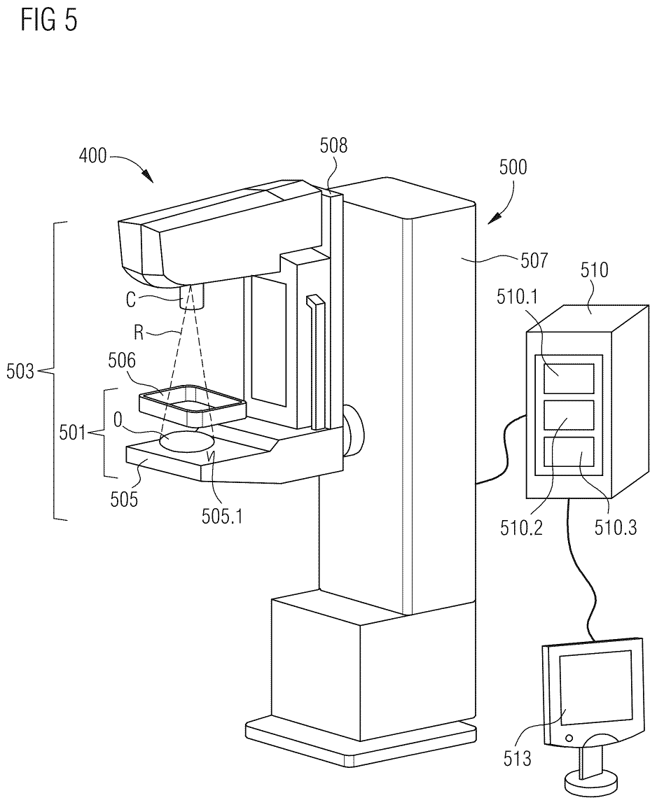

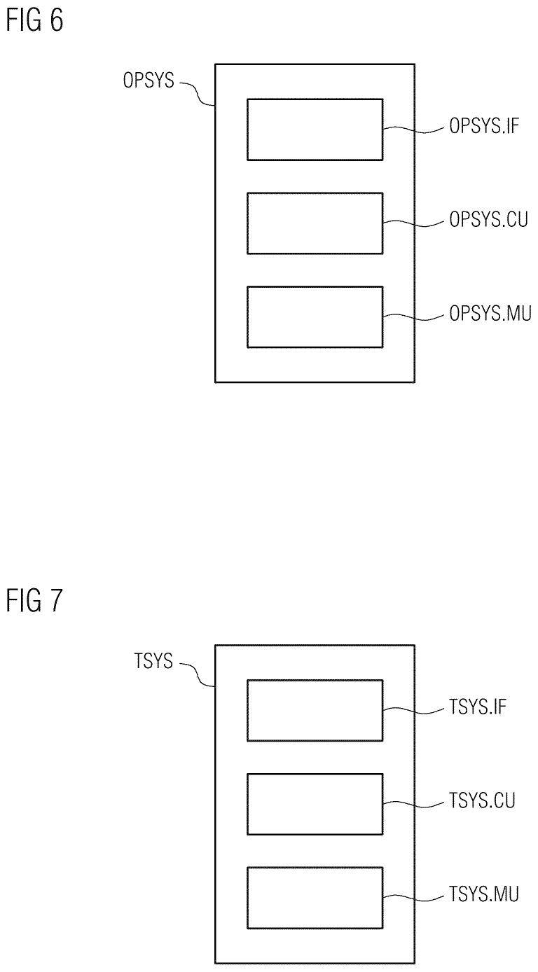

FIG. 6 shows an exemplary embodiment of an optimization system OPSYS for providing a control sequence, FIG. 7 shows a training system TSYS for providing a trained function.

The optimization system OPSYS shown is designed so as to perform a method according to one or more example embodiments for providing a control sequence. The training system TSYS shown is designed so as to perform a method according to one or more example embodiments for providing the trained function. The optimization system OPSYS comprises an optimization interface OPSYS.IF, an optimization computing unit OPSYS.CU and an optimization storage device unit OPSYS.MU. The training system TSYS comprises a training interface TSYS.IF, a training computing unit TSYS. CU and a training storage device unit TSYS.MU.

The optimization system OPSYS and/or the training system TSYS can be in particular a computer, a micro-controller or an integrated circuit (IC). Alternatively, the optimization system OPSYS and/or the training system TSYS can be a real or virtual computer network (a technical term for a real computer network is ‘cluster’, a technical term for a virtual computer network is ‘cloud’). The optimization system OPSYS and/or the training system TSYS can be designed as a virtual system running on a computer or a real computer network or a virtual computer network, (a technical term for this is ‘virtualization’).

The optimization interface OPSYS. IF and/or the training interface TSYS. IF can be a hardware or software interface, (for example as a PCI bus, CAN bus, USB or Firewire). The optimization computing unit OPSYS. CU and/or the training computing unit TSYS. CU can comprise hardware and/or software components, for example a micro-processor or a so-called FPGA (field programmable gate array). The optimization storage device unit OPSYS. MU and/or the training storage device unit TSYS.MU can be designed as a non-permanent working memory (random access memory, RAM) or as a permanent mass storage device (hard drive, USB stick, SD card, solid state disk (SSD)).

The optimization interface OPSYS. IF and/or the training interface TSYS. IF can in particular comprise a plurality of sub-interfaces that perform different method steps of the respective method according to one or more example embodiments. In other words, the optimization interface OPSYS. IF and/or the training interface TSYS. IF can be designed as a plurality of optimization interfaces OPSYS. IF and/or training interfaces TSYS. IF. The optimization computing unit OPSYS.CU and/or the training computing unit TSYS. CU can in particular comprise a plurality of sub-computing units that perform different method steps of the respective method according to one or more example embodiments. In other words, the optimization computing unit OPSYS. CU and/or the training interface TSYS. CU can be designed as a plurality of optimization computing units OPSYS.UC and/or training computing units TSYS. CU.

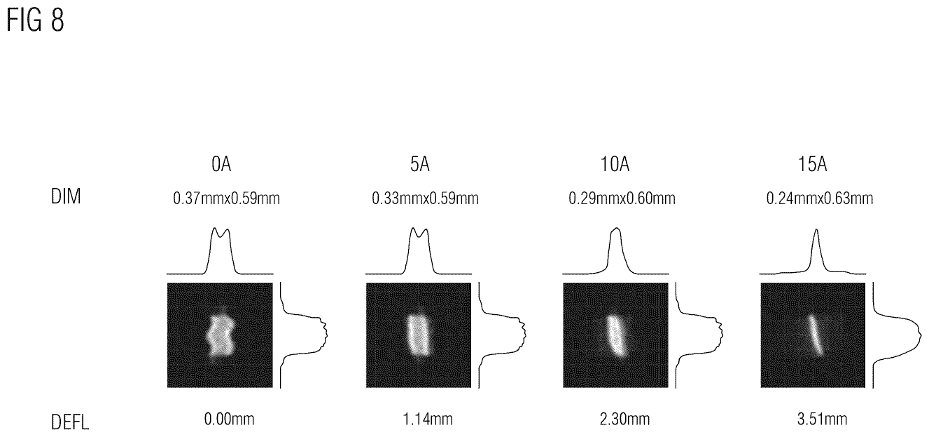

FIG. 8 shows an exemplary embodiment of a map of dimensions DIM of a focal spot 430.

The images of the focal spot can be determined using the method described in the IEC60336 standard. They display the projection of the focal spot on a reference plane perpendicular to the direction of observation. The direction of observation hereby connects the focal spot to the pinhole aperture used for the measurement and the detector used. The reference plane intersects the focal spot as described in IEC6336 on the anode. The dimensions DIM shown are merely an example for the optical dimensions of the focal spot on the anode.

Different dimensions DIM of the focal spot 430 on the anode 412 are illustrated for different deflection values. In this exemplary embodiment, the deflection values are deflection currents which can be applied to a deflection unit 417a, 417b, in particular an air coil, for deflecting the electron beam.

In addition, a spatial deflection DEFL of the focal spot 430 on the anode 412 can be indicated. In this case, the deflection DEFL relates to a zero position of the focal spot 430 on the anode 412. The zero position is the position at which the electron beam when not deflected strikes the anode 412.

The map applies for a fixed acceleration voltage. The acceleration voltage is the voltage which is applied between the cathode 411 and the anode 412 in the X-ray source 400 in order to accelerate electrons or the electron beam onto the anode 412. The dimensions DIM and the deflection DEFL from the zero position is dependent upon the acceleration voltage and the tube current or the current applied at the cathode or the emitter.

According to the map, the shape of the focal spot 430 rotates slightly with an increasing deflection DEFL. This can be explained by the shape of the anode 412. In the case of a deflection DEFL in the other direction from the perspective of the zero position (not shown in the map), the rotation would be precisely in the opposite direction due to the geometry of the anode 412.

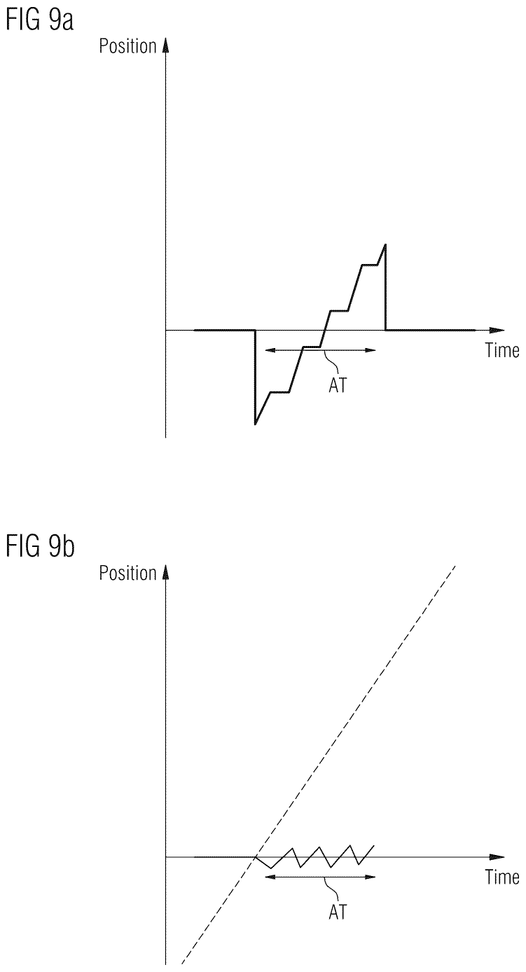

FIG. 9a shows a first exemplary embodiment of a movement of a focal spot 430 on an anode 412.

The diagram shows a position of the focal spot 430 on the anode 412 of an X-ray source 400 during an exposure period AT.

The zero position in this illustration is the position at which the time axis intersects the position axis. The zero position on the focal spot 430 on the anode 412 is the position at which the electron beam when not deflected strikes the anode 412.

In the exemplary embodiment shown, the focal spot 430 assumes four discrete positions on the anode during the exposure period AT. The electron beam is therefore deflected along four different deflection angles. In other words, the control sequence comprises four deflection values for deflecting the electron beam according to the four deflection angles. The focal spot 430 jumps between the four discrete positions. Alternatively, the focal spot is moved at least very quickly from one position to the next position. Alternatively, the focal spot 430 can be moved between the discrete positions along a curve, for example a sinusoidal curve. The alternative embodiment for the movement between the discrete deflection values can also be transferred to the examples below.

In alternative embodiments, the focal spot 430 can assume more or less than four discrete positions during the exposure period AT. In particular, the number of discrete stages at least of the distance which the X-ray source 400 travels during the exposure period can be divided by approximately half the average width of the focal spot 430. The width of the focal spot 430 corresponds to the dimension into which the focal spot can be moved.

An interpolated incline between the discrete positions can correspond in particular to a speed of a planned movement of the X-ray source 400.

In particular, the discrete positions can be selected in such a manner that particularly favorable positions would be selected according to the map of the dimensions DIM of the focal spot 430

FIG. 9b shows a resulting movement of the focal spot 430 from the perspective of an X-ray detector 505 according to the first exemplary embodiment from FIG. 9a.

A planned continuous movement of the X-ray source 400 is represented in the form of the dashed straight lines. The incline of the straight lines corresponds to the speed at which the X-ray source 400 is moved.

The diagram shows how the focal spot 430 moves during the exposure period AT from the perspective of a fixed X-ray detector 505. Alternatively, the diagram can also represent the perspective of an examination object.

While the focal spot 430 assumes a fixed position on the anode 412 according to FIG. 9a, the focal spot 430 moves at the speed of the X-ray source 400 from the perspective of the X-ray detector 505. In this exemplary embodiment, the positions of the focal spot 430 on the anode are selected in such a manner that from the perspective of the X-ray detector the focal spot 430 jumps back into its original position after a certain time.

It is possible in this manner to reduce smudging of the focal spot 430 from the perspective of the X-ray detector and consequently the effective focus size to at least a quarter in this specific example with four discrete positions. By suitably selecting the positions of the focal spot 430 in dependence upon the map, it is possible to further reduce the effective focus size.

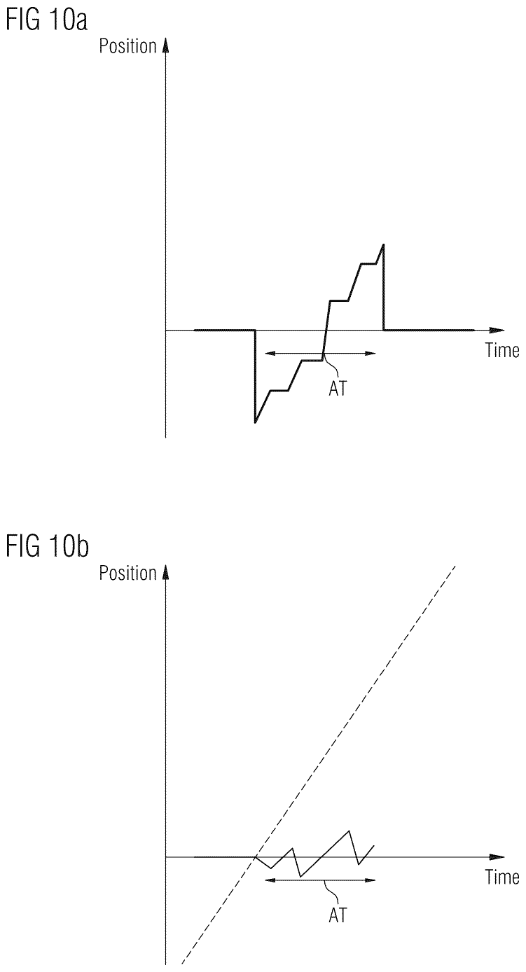

FIG. 10a shows a second exemplary embodiment of a movement of a focal spot 430 on an anode 412.

The diagram shows a position of the focal spot 430 on the anode 412 of an X-ray source 400 during an exposure period AT.

The zero position in this illustration is the position at which the time axis intersects the position axis. The zero position of the focal spot 430 on the anode 412 is the position at which the electron beam when not deflected strikes the anode 412.

In the exemplary embodiment shown, the focal spot 430 assumes four discrete positions on the anode during the exposure period AT. The electron beam is therefore deflected along four different deflection angles. In other words, the control sequence comprises four deflection values for deflecting the electron beam according to the four deflection angles. The focal spot 430 jumps between the four discrete positions. Alternatively, the focal spot 430 is moved at least very quickly from one position to the next position.

An interpolated incline between the discrete positions can correspond in particular to a speed of a planned movement of the X-ray source 400.

According to the exemplary embodiment, the four discrete positions are not equidistant from one another. In particular, a region around the zero position of the focal spot 430 on the anode 412 is ignored or skipped. It is clear from the map in FIG. 8 that in the region of the zero position, i.e. in the region which the electron beam when not deflected strikes on the anode 412, the focal spot 430 is especially large.

FIG. 10b shows a resulting movement of the focal spot from the perspective of an X-ray detector according to the second exemplary embodiment from FIG. 10a.

A planned continuous movement of the X-ray source 400 is represented in the form of the dashed straight lines. The incline of the straight lines corresponds to the speed at which the X-ray source 400 is moved.

The diagram shows how the focal spot 430 moves during the exposure period AT from the perspective of a fixed X-ray detector 505. Alternatively, the diagram can also represent the perspective of an examination object.

By skipping the zero position according to FIG. 10a, the focal spot 430 is blurred to a greater extent from the perspective of the X-ray detector, in other words the effective focus. However, by “skipping” the region of the large focal spot 430 on the anode 412, the effective focus shape can be further optimized, since the in deflected regions the focal spot 430 is narrower and consequently smudging is not so serious.

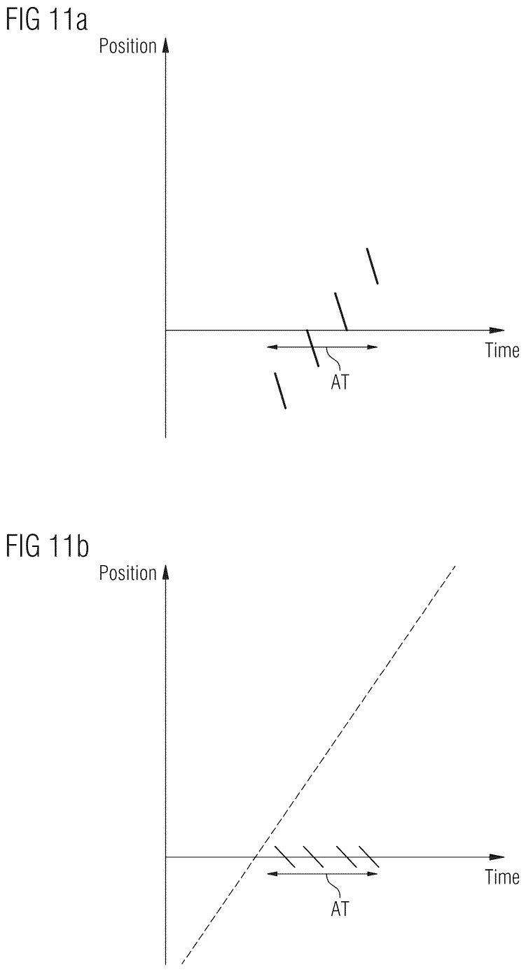

FIG. 11a shows a third exemplary embodiment of a movement of a focal spot 430 on an anode 412.

The diagram shows a position of the focal spot 430 on the anode 412 of an X-ray source 400 during an exposure period AT.

The zero position in this illustration is the position at which the time axis intersects the position axis. The zero position on the focal spot 430 on the anode 412 is the position at which the electron beam when not deflected strikes the anode 412.

According to the exemplary embodiment, the focal spot 430 on the anode 412 moves during the exposure period AT with a planned movement of the X-ray source 400 between respectively two discrete positions of the focal spot. In particular, the speed of the movement of the focal spot 430 can be quicker than the movement of the X-ray source 400. Between them, the focal spot 430 jumps to different regions of the anode 412.

FIG. 11b shows a resulting movement of the focal spot 430 from the perspective of an X-ray detector 505 according to the third exemplary embodiment from FIG. 11a.

A planned continuous movement of the X-ray source 400 is represented in the form of the dashed straight lines. The incline of the straight lines corresponds to the speed at which the X-ray source 400 is moved.

The diagram shows how the focal spot 430 moves during the exposure period AT from the perspective of a fixed X-ray detector 505. Alternatively, the diagram can also represent the perspective of an examination object.

From the perspective of the X-ray detector 505, the focal spot 430 moves during a movement according to FIG. 11a quicker than the X-ray source 400. In this manner, the focal spot 430 of the X-ray source 400 runs quasi ahead and then “waits” for the X-ray source 400. It is possible in this manner to reduce smudging of the effective focus size by moving the X-ray source.

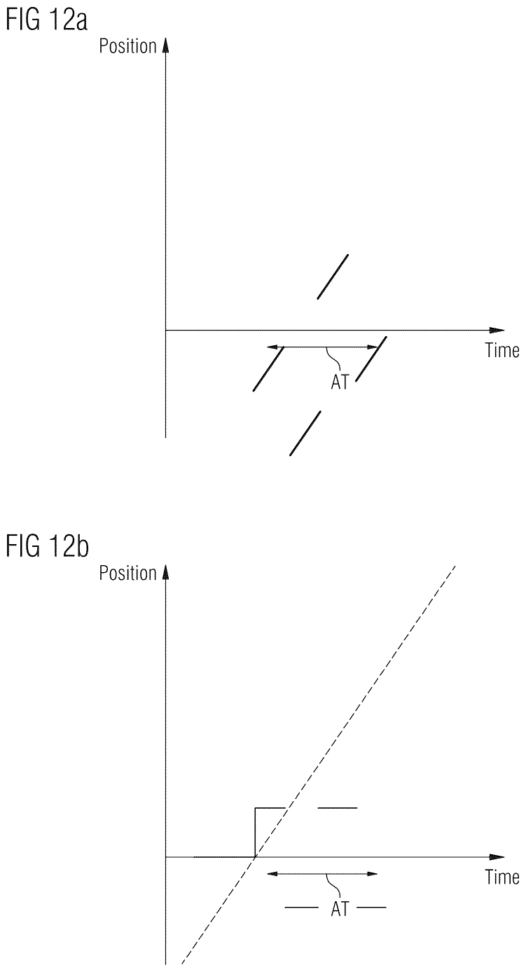

FIG. 12a shows a fourth exemplary embodiment of a movement of a focal spot 430 on an anode 412.

The diagram shows a position of the focal spot 430 on the anode 412 of an X-ray source 400 during an exposure period AT.

The zero position in this illustration is the position at which the time axis intersects the position axis. The zero position on the focal spot 430 on the anode 412 is the position at which the electron beam when not deflected strikes the anode 412.

According to the exemplary embodiment, the focal spot 430 is moved to different regions of the anode 412 in the opposite direction to the movement of the X-ray source 412. The regions in this case are advantageously adjacent to the zero position of the focal spot 430 on the anode 412.

In this manner, the movement of the X-ray source 412 in these regions is compensated by moving the focal spot 430 in the opposite direction.

The movement resembles that of a well-known spring focus for computer tomography. The movement was only slightly modified for use in tomosynthesis.

FIG. 12b shows a resulting movement of the focal spot 430 from the perspective of an X-ray detector 505 according to the fourth exemplary embodiment from FIG. 12a.

A planned continuous movement of the X-ray source 400 is represented in the form of the dashed straight lines. The incline of the straight lines corresponds to the speed at which the X-ray source 400 is moved.

The diagram shows how the focal spot 430 moves during the exposure period AT from the perspective of a fixed X-ray detector 505. Alternatively, the diagram can also represent the perspective of an examination object.

According to the exemplary embodiment, the focal spot 430 jumps from the perspective of the X-ray detector 505 and consequently the effective focus between two discrete points. The effective focus remains then at these discrete points for a part of the exposure period AT.

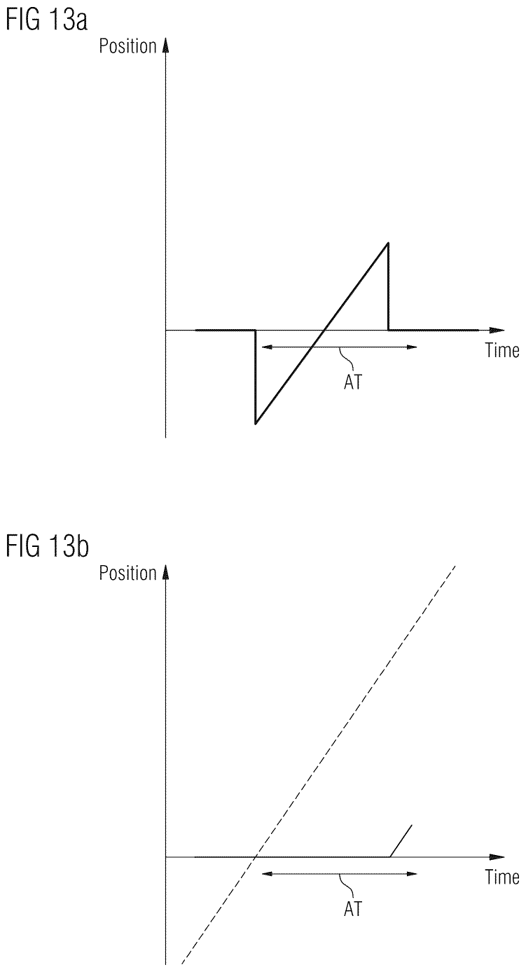

FIG. 13a shows a fifth exemplary embodiment of a movement of a focal spot 430 on an anode 412.

The diagram shows a position of the focal spot 430 on the anode 412 of an X-ray source 400 during an exposure period AT.

The zero position in this illustration is the position at which the time axis intersects the position axis. The zero position on the focal spot 430 on the anode 412 is the position at which the electron beam when not deflected strikes the anode 412.

According to the exemplary embodiment, the focal spot 430 is moved during a part of the exposure period AT in the opposite direction to the planned movement of the X-ray source 400. The speed of the movement of the focal spot 430 corresponds in this case advantageously to the speed of the planned movement of the X-ray source 400.

The movement of the focal spot 430 ends prior to the end of the exposure period, whereas the X-ray source continues to move. Advantageously, the movement of the focal spot 430 ends in a position on the anode 412 at which the focal spot has an advantageous shape and smudging of the effective focus shape is not too serious.

It is possible in this manner in particular, for example, to prevent that the focal spot 430 is positioned in a region on the anode 412 at which it is greatly twisted or rotated, and the shape of the focal spot alone causes an enlargement of the effective focus from the perspective of the X-ray detector 505. In particular, it is possible to achieve reduced smudging of the focal spot 430 when a maximum deflection region of the deflection unit is smaller than the movement distance of the X-ray source 400 during an exposure period AT.

FIG. 13b shows a resulting movement of the focal spot from the perspective of an X-ray detector according to the fifth exemplary embodiment from FIG. 13a.

A planned continuous movement of the X-ray source 400 is represented in the form of the dashed straight lines. The incline of the straight lines corresponds to the speed at which the X-ray source 400 is moved.

The diagram shows how the focal spot 430 moves during the exposure period AT from the perspective of a fixed X-ray detector 505. Alternatively, the diagram can also represent the perspective of an examination object.

The focal spot 430 appears from the perspective of the X-ray detector 505 to be static, i.e. stationary, for a part of the exposure period AT. In other words, the effective focus shape during this part of the exposure period AT is not influenced by the movement of the X-ray source 400. During the time period in which the focal spot 430 is no longer moved in the opposite direction to the X-ray source 400, the focal spot 430 moves relative to the X-ray detector 505 at the speed of the X-ray source 400. This leads to a smudging of the effective focus shape, i.e. of the focal spot 430 from the perspective of the X-ray detector 505.

In particular, it is possible in this manner to reduce influences on the effective focus shape caused by a tilting or rotating of the focal spot 430 on the anode 412, since the focal spot 430 is not moved in regions on the anode 412 in which the focal spot 430 tilts. In particular, it is possible to achieve reduced smudging of the focal spot 430 when a maximum deflection region of the deflection unit is smaller than the movement distance of the X-ray source 400 during an exposure period AT.

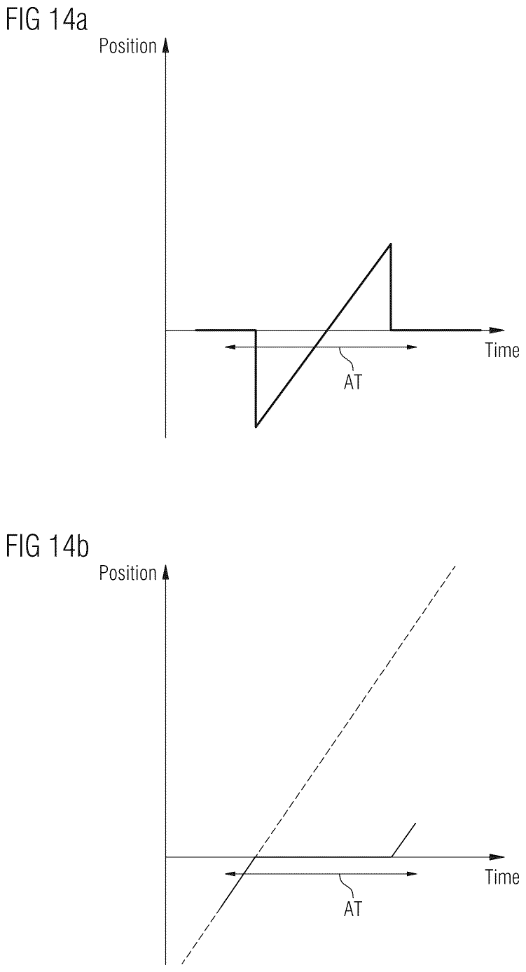

FIG. 14a shows a sixth exemplary embodiment of a movement of a focal spot 430 on an anode 412.

The diagram shows a position of the focal spot 430 on the anode 412 of an X-ray source 400 during an exposure period AT.

The zero position in this illustration is the position at which the time axis intersects the position axis. The zero position on the focal spot 430 on the anode 412 is the position at which the electron beam when not deflected strikes the anode 412.

As also in FIG. 13a, the focal spot 430 is moved at the speed of the planned movement of the X-ray source 400 in the opposite direction to this movement of the X-ray source 400.

According to the exemplary embodiment, the movement begins after commencement of the exposure period and ends prior to the end of the exposure period. In particular, the focal spot 430 can be moved during 30% of the exposure period AT or during 40% of the exposure period AT or during 50% of the exposure period AT or during 60% of the exposure period AT or during 70% of the exposure period AT or during 80% of the exposure period AT or during 90% of the exposure period AT in the opposite direction to the movement of the X-ray source 400. In particular, the focal spot 430 can be moved during 20% to 95% of the exposure period AT in the opposite direction to the movement of the X-ray source 400.

FIG. 14b shows a resulting movement of the focal spot from the perspective of an X-ray detector according to the sixth exemplary embodiment from FIG. 14a.

A planned continuous movement of the X-ray source 400 is represented in the form of the dashed straight lines. The incline of the straight lines corresponds to the speed at which the X-ray source 400 is moved.

The diagram shows how the focal spot 430 moves during the exposure period AT from the perspective of a fixed X-ray detector 505. Alternatively, the diagram can also represent the perspective of an examination object.

The focal spot 430 appears from the perspective of the X-ray detector to be static, i.e. stationary, for a part of the exposure period AT. In other words, the effective focus shape during this part of the exposure period AT is not influenced by the movement of the X-ray source 400. During the time period in which the focal spot 430 is no longer moved in the opposite direction to the X-ray source 400, the focal spot 430 moves relative to the X-ray detector 505 at the speed of the X-ray source 400. This leads to a smudging of the effective focus shape, i.e. of the focal spot 430 from the perspective of the X-ray detector 505.

In particular, it is possible in this manner to reduce influences on the effective focus shape caused by a tilting or rotating of the focal spot 430 on the anode 412, since the focal spot 430 is not moved in regions on the anode 412 in which the focal spot 430 tilts. In particular, boundary regions of the deflection of the electron beam are ignored in order to minimize an influence of a tilting or rotating of the focal spot 430. In particular, it is possible to achieve reduced smudging of the focal spot 430 when a maximum deflection region of the deflection unit is smaller than the movement distance of the X-ray source 400 during an exposure period AT.

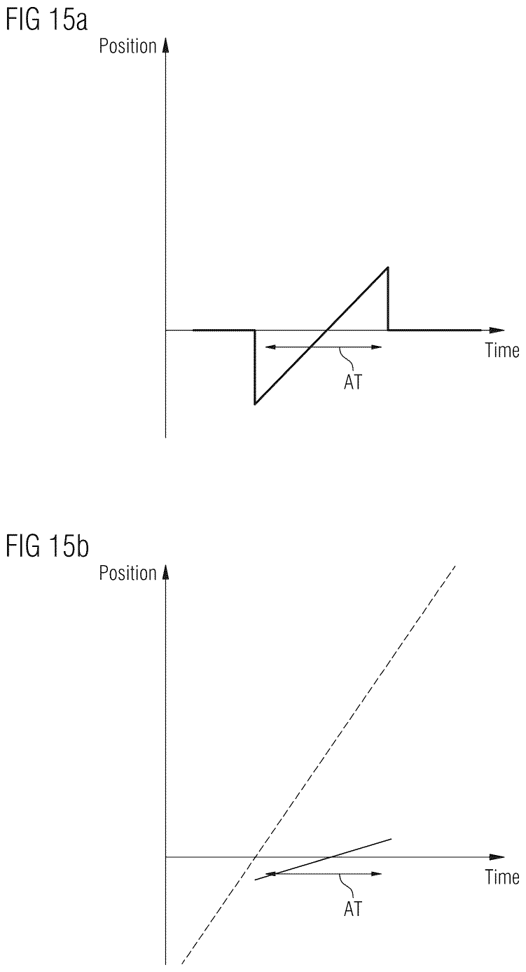

FIG. 15a shows a seventh exemplary embodiment of a movement of a focal spot 430 on an anode 412.

The diagram shows a position of the focal spot 430 on the anode 412 of an X-ray source 400 during an exposure period AT.

The zero position in this illustration is the position at which the time axis intersects the position axis. The zero position on the focal spot 430 on the anode 412 is the position at which the electron beam when not deflected strikes the anode 412.

In the exemplary embodiment, the focal spot 430 is moved at a speed slower than the speed of the planned movement of the X-ray source 400 in the opposite direction to this planned movement of the X-ray source 400. In particular, it is consequently possible to achieve reduced smudging of the focal spot 430 when a maximum deflection region of the deflection unit is smaller than the movement distance of the X-ray source 400 during an exposure period AT.

Optionally, the movement of the focal spot 430 can begin after the commencement of the exposure period AT and/or end prior to the end of the exposure period AT. In other words, the focal spot 430 can only be moved as described during a part of the exposure period AT.

FIG. 15b shows a resulting movement of the focal spot 430 from the perspective of an X-ray detector 505 according to the sixth exemplary embodiment from FIG. 15a.

A planned continuous movement of the X-ray source 400 is represented in the form of the dashed straight lines. The incline of the straight lines corresponds to the speed at which the X-ray source 400 is moved.

The diagram shows how the focal spot 430 moves during the exposure period AT from the perspective of a fixed X-ray detector 505. Alternatively, the diagram can also represent the perspective of an examination object.

From the perspective of the X-ray detector, the focal spot moves in the direction of the X-ray source 400 but more slowly than the latter. It is possible in this manner to optimize smudging of the focal spot 430 from the perspective of the X-ray detector 505 and consequently the effective focus shape.

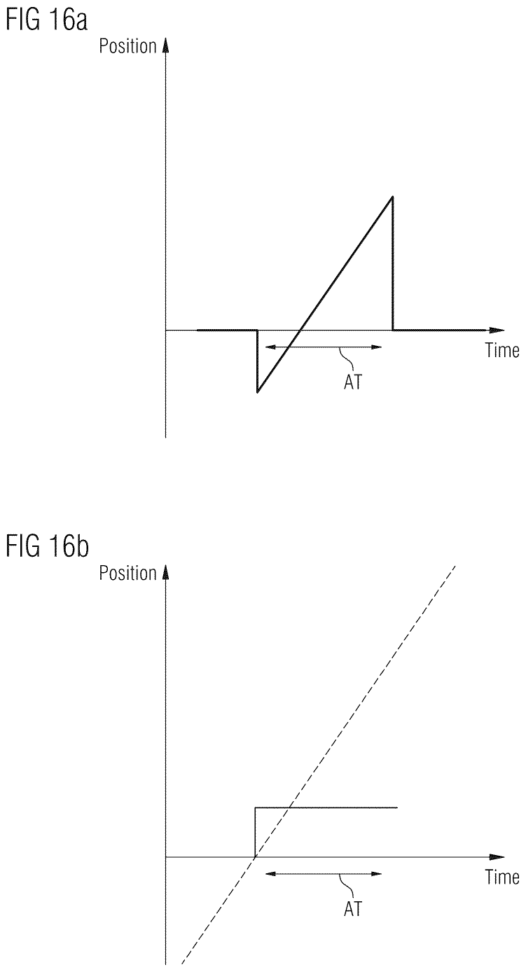

FIG. 16a shows an eighth exemplary embodiment of a movement of a focal spot 430 on an anode 412.

The diagram shows a position of the focal spot 430 on the anode 412 of an X-ray source 400 during an exposure period AT.

The zero position in this illustration is the position at which the time axis intersects the position axis. The zero position on the focal spot 430 on the anode 412 is the position at which the electron beam when not deflected strikes the anode 412.

According to the exemplary embodiment, the focal spot 430 is moved in an opposite direction to the movement of the X-ray source 400 at the speed of the movement of the X-ray source 400. In this case, the focal spot 430 is located outside or adjacent to the zero position on the anode.

Optionally, the movement of the focal spot 430 in this exemplary embodiment can also only take place during a part of the exposure period AT. In other words, the movement of the focal spot 430 can begin after the commencement of the exposure period AT and/or end prior to the end of the exposure period AT.

FIG. 16b shows a resulting movement of the focal spot 430 from the perspective of an X-ray detector 505 according to the eighth exemplary embodiment from FIG. 16a.

A planned continuous movement of the X-ray source 400 is represented in the form of the dashed straight lines. The incline of the straight lines corresponds to the speed at which the X-ray source 400 is moved.

The diagram shows how the focal spot 430 moves during the exposure period AT from the perspective of a fixed X-ray detector 505. Alternatively, the diagram can also represent the perspective of an examination object.

From the perspective of the X-ray detector 505, the focal spot 430 is stationary while it is moved on the anode 412. However, the focal spot is displaced in comparison to its zero position.

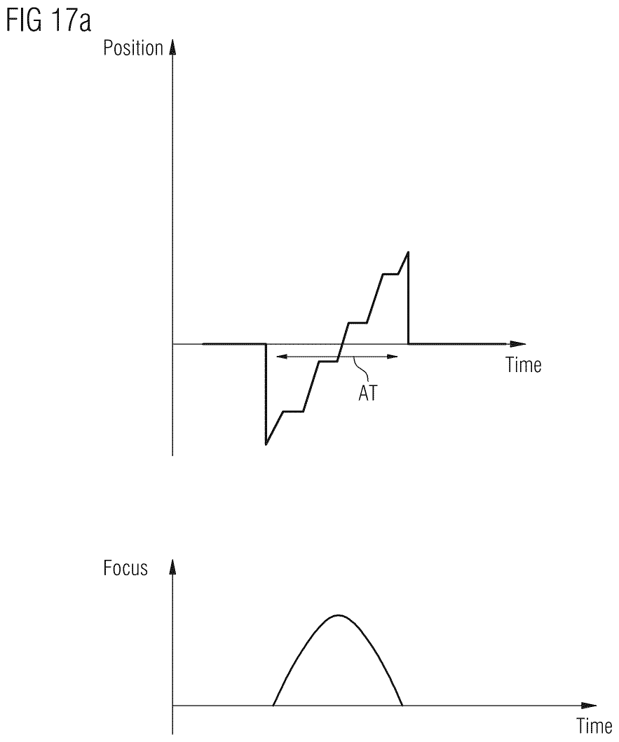

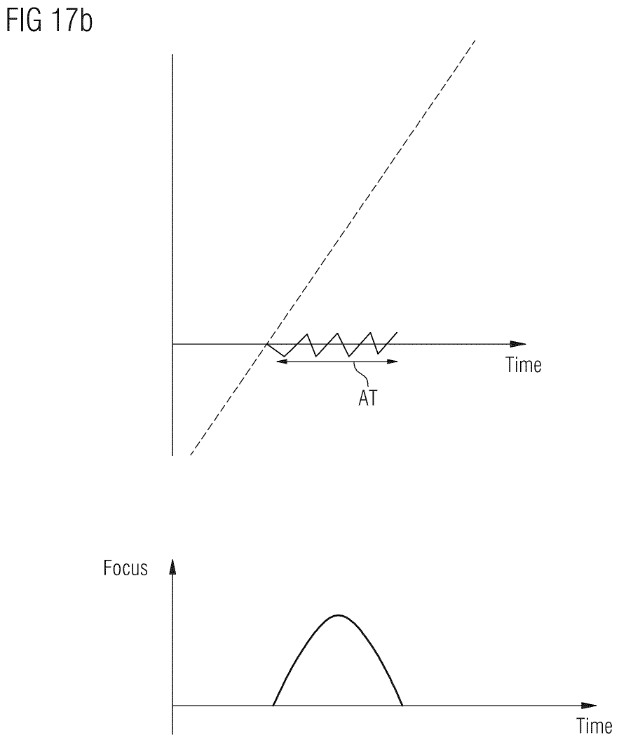

FIG. 17a shows a first exemplary embodiment according to FIG. 9a of a movement of a focal spot on an anode with a variable focusing,

The lower diagram of the illustration shows a strength of the focusing of the focal spot 430 during the exposure period AT. The strength of the focusing increases around the zero position of the focal spot 430 on the anode 412. In other words, the focal spot 430 becomes increasingly focused the closer it is positioned to the zero position.

In this manner, a widening of the focal spot 430, as shown in the map from FIG. 8, toward the zero position can be counteracted.

FIG. 17b shows a resulting movement of the focal spot from the perspective of an X-ray detector according to the first exemplary embodiment with a variable focusing from FIG. 17a.

The lower diagram also shows here the variable strength of the focusing analogous to FIG. 17a.

Where not already explicitly stated, but where it makes sense and is in line with the invention, individual exemplary embodiments, individual sub-aspects or features thereof may be combined or exchanged without departing from the scope of the present invention. Advantages of the invention described with reference to one exemplary embodiment also apply to other exemplary embodiments where transferable, without explicit mention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, module may or cloud) accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units various that perform operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References s to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for providing image data, the method comprising:
generating X-ray radiation via an X-ray source, the X-ray source including a cathode configured to generate an electron beam and an anode, the electron beam striking the anode forms a focal spot, the focal spot is moved on the anode during an exposure period by deflecting the electron beam on the anode, the deflection is based on a predetermined control sequence, such that the movement of the focal spot is not continuous in sections;
detecting X-ray radiation via an X-ray detector during the exposure period; and
providing the image data based on the detected X-ray radiation.

2. The method of claim 1, wherein the control sequence comprises discrete deflection values.

3. The method of claim 1, wherein the control sequence specifies at least one of a type or a speed the generation of the electron beam.

4. The method of claim 1, wherein the control sequence comprises two maximum deflection values.

5. The method of claim 4, wherein the control sequence comprises at least one further deflection value between the two maximum deflection values.

6. The method of claim 1, wherein the X-ray source is moved during the exposure period according to a planned movement.

7. The method of claim 6, wherein a speed of the movement of the focal spot generated by the deflection of the electron beam is different at least during a part of the exposure period than a speed of the planned movement of the X-ray source.

8. The method of claim 6, wherein the movement of the focal spot is performed at least partially in a direction of the planned movement of the X-ray source.

9. The method of claim 6, wherein
the control sequence comprises at least four different deflection values,
the control sequence comprises each of the deflection values twice,
the electron beam is deflected such that the electron beam is moved continuously between at least two of the deflection values following one another according to the control sequence opposite a direction of the movement of the X-ray source, and
the electron beam is deflected such that the electron beam jumps between at least two further deflection values following one another according to the control sequence in the direction of the movement of the X-ray source.

10. The method of claim 6, wherein during the movement of the X-ray source the method is repeated for at least two exposure periods.

11. The method of claim 6, wherein the planned movement of the X-ray source is continuous.

12. The method of claim 1, wherein
the control sequence comprises at least two discrete deflection values following one another, and
the electron beam is deflected such that the electron beam jumps between the at least two discrete deflection values following one another according to the control sequence.

13. The method of claim 1, wherein the electron beam is deflected such that the movement of the focal spot is at least partially continuous.

14. The method of claim 1, further comprising:
focusing the electron beam around a zero position of the focal spot via a focusing unit.

15. The method of claim 14, wherein, the focusing focuses the electron beam less, the further away the focal spot is moved from the zero position by deflecting the electron beam.

16. A computer-implemented method for providing a control sequence, the method comprising:
receiving a map of dimensions of a focal spot, the focal spot formed by an electron beam striking an anode of an X-ray source, the dimensions being based on a deflection of the electron beam; and
determining a control sequence for deflecting the electron beam based on the map, the control sequence is determined such that an effective focus shape of X-ray radiation emitted from the focal spot is optimized over an exposure period with respect to the dimensions of the focal spot; and
providing the control sequence.

17. The method of claim 16, further comprising:
using the control sequence in method for providing image data.

18. The method of claim 16, further comprising:
receiving a planned movement of the X-ray source during the exposure period, wherein the determining the control sequence determines the control sequence based on the planned movement of the X-ray source.

19. The method of claim 16, wherein the determining the control sequence comprises:
applying a trained function to the map and a planned movement of the X-ray source, the determining the control sequence determines the control sequence based on the applying.

20. The method of claim 16, wherein the determining the control sequence comprises:
creating an optimization function based on the map and a planned movement of the X-ray source, the determining the control sequence determines the control sequence based on the optimization function.

21. A computer-implemented method for providing a trained function for determining a control sequence for deflecting an electron beam, the method comprising:
receiving training input data, the training input data including a map of dimensions of a focal spot, the focal spot formed by an electron beam striking an anode of an X-ray source, the dimensions are based on a deflection of the electron beam, the training input data;
receiving training output data, the training output data including control sequences with which optimized effective focus shapes of X-ray radiation emitted from the focal spot are achievable;
training the trained function based on the training input data and the training output data; and
providing the trained function.

22. An optimization system comprising:
an interface; and
a computing unit, wherein the interface and the computing unit are configured to cooperatively perform the method of claim 16.

23. An X-ray source configured to perform the method of claim 1.

24. The X-ray source of claim 23, further comprising:
an X-ray radiation unit comprising the cathode and the anode;
a deflection unit; and
a single tank in which the X-ray radiation unit and the deflection unit are arranged, wherein
the X-ray radiation unit is enclosed by a vacuum housing, and
the deflection unit configured to deflect the electron beam in the X-ray unit according to the control sequence.

25. The X-ray source of claim 24, wherein the deflection unit comprises an air coil.

26. The X-ray source of claim 23, further comprising:
an X-ray radiation unit comprising the cathode, the anode and a deflection unit; and
a single tank in which the X-ray radiation unit is arranged, wherein
the X-ray radiation unit is enclosed by a vacuum housing, and
the deflection unit configured to deflect the electron beam in the X-ray unit according to the control sequence.

27. The X-ray source of claim 26, wherein the deflection unit deflects the electron beam via electric or magnetic deflection.

28. An imaging system comprising:
an interface;
a computing unit;
an X-ray source; and
an X-ray detector, wherein the interface, the computing unit, the X-ray source and the X-ray detector are configured to cooperatively perform the method of claim 1.

29. The imaging system of claim 28, wherein the imaging system is a mammography system further comprising:
a compression unit configured to compress an examination object, wherein the compression unit is arranged between the X-ray source and the X-ray detector.

30. The imaging system of claim 29, wherein the X-ray source is pivotable relative to at least one of the X-ray detector or the compression unit to perform a planned movement.
